(19) United States
(12) Patent Application Publication
(10) Pub. No.: US 2002/0137209 A1
(43) Pub. Date: Sep. 26, 2002

(54) Methods of dissociating nongenotropic from genotropic activity of steroid receptors

(76) Inventors: Stavroula Kousteni, Little Rock, AR (US); Stavros C. Manolagas, Little Rock, AR (US)

Correspondence Address: KING & SPALDING, 191 PEACHTREE STREET, N.E., ATLANTA, GA, 30303-1763, US

(21) Appl. No.: 09/880,710

(22) Filed: Jun. 13, 2001

Related U.S. Application Data
(60) Provisional application No. 60/211,287, filed on Jun. 13, 2000.
(60) Provisional application No. 60/274,373, filed on Mar. 8, 2001.

(51) Int. Cl. C12N 15/85; C07K 14/72

(52) U.S. Cl. 435/455; 530/350

(57) ABSTRACT

This invention concerns the fundamental discovery of the understanding of the mechanism of action of steroidal nongenotropic effects and their relation to steroidal genotropic effects. It has been discovered that (i) steroidal nongenotropic effects and genotropic effects can be mediated by the same steroid receptor; (ii) both effects are ligand-induced; (iii) the nongenotropic effect occurs due to a ligand-induced activation of the ligand binding domain, which can be fast and loose fitting; (iv) the genotropic effect occurs due to a ligand-induced activation of the DNA-binding domain of the steroid receptor, which is typically a result of a slower, stronger interaction; and (v) the nongenotropic effect of a ligand interaction can be dissociated from the genotropic effect of a ligand interaction, in such a manner to effect a selective response. This invention also describes, for the first time, that certain steroids are capable of inducing a nongenotropic effect via binding to an unrelated steroid receptor

### FEDERAL FUNDING

This invention was funded in part through a grant from the U.S. National Institutes of Health. Therefore, the U.S. federal government has certain rights in this invention.

This application claims priority to U.S. Ser. No. 60/211,287, entitled, “Sex Non-Specific Methods and Compositions for Increasing Bone Mass”, filed on Jun. 13, 2000 and U.S. Ser. No. 60/274,373, entitled “Methods of Dissociating Nongenotropic from Transcriptional Activity of Steroid Receptors”, filed on Mar. 8, 2001.

### FIELD OF THE INVENTION

This invention is in the field of steroid signalling, and in particular describes a method of dissociating a steroidal nongenotropic effect from the steroidal genotropic effect, and a method for screening compounds that are capable of inducing a steroidal nongenotropic effect without substantially inducing a steroidal genotropic effect or are capable of inducing a steroidal genotropic effect without substantially inducing a steroidal nongenotropic effect. This invention also describes, for the first time, that certain steroids are capable of inducing a nongenotropic effect via binding to an unrelated steroid receptor.

### BACKGROUND OF THE INVENTION

Due to similarities in molecular structure and homologies in protein sequence, a number of proteins have been classified into a superfamily of steroid nuclear receptors. The steroid protein receptor family includes at least sixty four known receptors (Cell, Vol 97, 161-163, Apr. 16, 1999), including the α and β form of the estrogen receptors (ERα and ERβ), the estrogen receptor related receptor-1 and receptor-2 (ERR-1 and ERR-2), the androgen receptor (AR), the progesterone receptor (PR), the retinoic acid receptor (and related orphan receptors ROR-α, ROR-β, and ROR-γ); the glucocorticoid receptor (GR), the mineralcorticoid receptor (MR), the Vitamin D receptor, the neuroactive receptor, the famesoid X receptor (FXR), the liver X receptor (LXRα and LXRβ), the thyroid hormone receptors A and B (which bind triiodothyronine (T3) and thyroxine (T4)), the COUP-TF receptor, the ecdysone receptor, the peroxisome proliferator activated protein receptors (PPAR, including PPARα, PPARγ, and PPARδ), pregnane X (PXR), the bile acid binding family, as well as chimeric receptors and orphan receptors.

It has traditionally been accepted that steroids bind to intracellular receptors and subsequently modulate transcription and protein synthesis typically via a steroid/receptor complex that induces gene activity. Even though the steroid receptors are primarily known as ligand-activated transcription factors, recently, a number of very rapid (e.g., seconds to approximately fifteen minutes) effects of steroids have been identified that are inconsistent with a genotropic effect by virtue of the rapidity of action. These observed nongenotropic effects of steroid hormones were recently reviewed by Falkenstein, et al., in “Multiple Actions of Steroid Hormones-A Focus on Rapid, Nongenotropic Effects,” Pharmacol. Rev 52:513-555, 2000. See also McEwen, B. S. and Alves, S. E., Endocr. Rev. 20, 279-307, 1999. Many of these rapid actions have been attributed to the ability of estrogens or other steroids to interact with putative membrane-associated receptors (Watson, et al., Proc. Soc. Exp. Biol. Med., 220, 9-19, 1999). However, heretofor it was unknown whether these nongenotropic effects are mediated by proteins that are the same as or distinct from those that mediate the transcriptional effects of the steroids. Moreover, the relationship of nongenotropic effects of steroids to the transcriptional activity of the classical receptors is completely unknown. For example, Falkenstein et al. made the following observations in their 2000 review article.

(i) Evidence for nongenotropic steroid effects and distinct receptors involved is presented for all steroid groups including related compounds like vitamin D, and thyroid hormones. The physiological and clinical relevance of these rapid effects is still largely unclear. . . . Falkenstein, et al., page 514, abstract.

(ii) In contrast to genotropic steroid action, nongenotropic steroid effects are principally characterized by their insensitivity to inhibitors of transcription and protein synthesis, and-representing the most obvious experimental evidence-by their rapid onset of action (within seconds to minutes). These rapid effects are likely to be mediated through receptors with pharmacological properties distinct from those of the intracellular steroid receptors . . . Discrepancies in pharmacological properties alone are not sufficient to support the hypothesis of separate receptor proteins for nongenotropic action; however, this important issue is addressed in Section III.B., and various evidence for the involvement of both classic and nonclassic receptor proteins in nongenotropic signaling is given. Falkenstein, et al., page 516.

(iii) A wide array of nongenotropic effects of steroids appear to be mediated through putative nonclassic membrane receptors with pharmacological properties that are clearly distinct from those of the classic intracellular steroid receptors. Falkenstein, page 520.

(iv) In summary, a substantial body of evidence now exists to indicate that at least some of the rapid 1α, 25-(OH)2D3-induced effects are transmitted by a membrane receptor distinct from the intracellular receptors belonging to the steroid and thyroid hormone superfamily. Falkenstein et al, page 521.

(v) It is obvious that many aspects of nongenotropic steroid action still require continuous intensive research, and essential clues for their understanding are still lacking. This also includes the still unaccomplished cloning of the putative membrane receptor initiating rapid steroid signaling. There are preliminary findings on membrane proteins that bind steroids (mPR, annexin II), but none has been definitively proven to represent a steroid membrane receptor responsible for rapid action. Clearly, the cloning or such a receptor would be a major advance, but it should not be forgotten that the definition of the physiological, pathophysiological, and last but not least, clinical relevance of novel aspects of steroid action should be another target of paramount importance. This may even be achieved without the receptors cloned, although the latter would definitively also ease clinically oriented research. Falkenstein et. al., page 546.

In particular, Falkenstein et al, included the following as evidence for putative separate receptors mediating the nongenotropic action of steroids:

(i) the existence of nongenotropic steroid effects in cells or tissues devoid of the respective classic receptor (e.g., in cells from knockout animals as shown for MR and PR) and the insensitivity of rapid steroid effects to classic antagonists (Falkenstein, et al., page 520); and

(ii) the observation that RU-28318 blocks rapid signaling does not necessarily imply that rapid effects of aldosterone are mediated by the classic receptor, an unlikely assumption given the fact that in MR knockout mice aldosterone is still nongenotropically active (Falkenstein, et al., page 535).

As a nonlimiting example of this, sex steroids have been used to treat bone loss but the underlying mechanism of action is unclear. The genotropic effects of sex steroids are mediated through nuclear receptor proteins, which by and large, discriminate between estrogen and androgen signaling and are distributed in a sex specific pattern among female and male target organs, respectively. Besides the classical estrogen receptor (ERα), many tissues express a second form of the estrogen receptor (ERβ). Binding of the steroids to their respective cognate receptors leads to conformational changes of the protein which allow it to interact with the transcriptional machinery directly, or indirectly via protein--protein interactions with other transcription factors. Numerous effects of estrogen have been documented over the last few years in a variety of cell types that exhibit such a fast action that it makes a genotropic mechanism of action unlikely (Lieberherr, M., B. et al. (1993) J. Bone Miner.Res. 8:1365-1376; Aronica, S. M. et al. (1994) Proc Natl.Acad.Sci.U.S.A. 91:8517-8521; Endoh, H., H. et al. (1997) Biochem.Biophys.Res.Commun. 235-99-102; McEwen, B. S. and S. E. Alves. (1999) Endocr.Rev. 20:279-307; Chen, Z., et al. (1999) J.Clin.Invest. 103:401-406; Castoria, G. et al. (1999) EMBO J. 18:2500-2510; Toran-Allerand, C. D. et al. (1999) Front. Neuroendocrinol. 20:97-121; Norfleet, A. M. et al. (1999) Endocrinology 140:3805-3814). Many of these rapid actions have been attributed to the ability of estrogen to interact with a membrane-associated form of ER (Collins, P. and C. Webb. (1999) Nat.Med. 5:1130-1131; Pietras, R. J. and C. M. Szego. (1999) Nat.Med. 5:1330; Razandi, M. et al. (1999) MolEndocrinol. 13:307-319; Norfleet, A. M., et al. (1999) Endocrinology 140:3805-3814; Watson, C. S. and B. Gametchu (1999) ProcSoc.Exp.Biol.Med. 220:9-19). However, the precise identity and subcellular localization of this site, including whether it is the classical receptor or whether it is localized in the membrane bilayer, or the membrane structures called caveolae (Okamoto, T., A. et al. (1998) J.Biol.Chem. 273:5419-5422; Schlegel, A., et al. (1999) J.Biol.Chem. 274:33551-33556), or the cytosol, has not been determined.

### The Role of the ER and AR in Adult Bone Homeostasis

Receptors for ERα and ERβ or AR (androgen receptor) have been convincingly demonstrated in proliferating chondrocytes, osteoblasts, bone marrow stromal cells (Eriksen, E. F. et al. (1988) Science 241-84-86; Komm, B. S. et al. (1988) Science 241-81-84; Ernst, M. et al. (1991) Mol.Endocrinol. 5:1597-1606; Benz, D. J. et al. (1991) J.BoneMiner.Res. 6:531-541; Benz, D. J., et al. (1991) Endocrinology 128:2723-2730; Monaghan, B. A. et al. (1992) Clin.Orthop. 277-280; Bellido, T., G. et al. (1993) Endocrinology 133:553-562; Ben-Hur, H., G. et al. (1993) Calcif.Tissue Int. 53:91-96; Kusec, V., et al. (1998) J Clin.Endocrinol.Metab. 83:2421-2428; Kennedy, J. et al. (1999) Bone 24:9-16; Nilsson, L. O. et al. (1999) J.Clin.Endocrinol.Metab. 84:370-373), as well as in osteoclasts and their progenitors (Oursler, M. J. et al. (1993) Ann.Med. 25:361-371). Remarkably, the level of expression of ERα, ERβ and AR in osteoblasts and osteoclasts is at least 10-fold lower compared to reproductive tissues. In addition, there is no gender specific distribution of the receptors in bone cells, as similar levels of ER or AR compared to female and male reproductive organs, have been found in cells from males and females (Benz, D. J. et al. (1991) J.BoneMiner.Res. 6:531-541; Benz, D. J., et al. (1991) Endocrinology 128:2723-2730; Komm, B. S. et al. (1988) Science 241-81-84; Braidman, I. et al. (2000) Bone 26:423-427). Moreover, there is seemingly conflicting evidence suggesting on one hand that estrogen is the bone active sex steroid in both the male and the female adult skeleton, while on the other hand non-aromatizable androgen can protect the female skeleton against the adverse effects of estrogen deficiency (Riggs, B. L. et al. (1998) J.Bone Miner.Res. 13:763-776; Vanderschueren, D. et al. (1998) Bone 23:391-394). Indeed, clinical observations of decreased bone mass in a male with mutant estrogen receptor (Smith, E. P. et al. (1994) N.Engl.J.Med. 331:1056-1061) and increased bone mass after treatment with estrogen in two males with P-450 aromatase deficiency (Carani, C. et al. (1997) N.Engl.J Med. 337:91-95; Bilezikian, J. P. et al. (1998) N.Engl.J Med. 339:599-603) have suggested that estrogen derived by peripheral aromatization of androgens are critical for the maintenance of bone mass in men as well (Riggs, B. L. et al. (1998) J.Bone Miner.Res. 13:763-776). However, in all three cases, the decreased bone mass and the increased levels of bone markers in young males with estrogen-deficiency, in the face of androgen sufficiency, could well be the result of failure to achieve peak bone mass because of defective skeletal growth during development as well as the fact that these individuals never underwent puberty—not to loss of bone mass, as it is the case with the common forms of osteoporosis. In support of this contention, humans with complete androgen insensitivity, due to mutations in the androgen receptor gene on the X-chromosome, have decreased bone mass, in spite of the elevated estrogen levels Schwartz, B. D. et al. (1999) Endrocrine Society 81^{st} Annual Meeting 93(Abstr)). Moreover, androgens, including non-aromatizable ones, like DHT, have identical effects to those of estrogen on the biosynthetic activity and the birth as well as the death of bone cells in vitro and in vivo, at least in rodents (Bellido, T. et al. (1995) J. Clin. Invest. 95:2886-2895; Weinstein, R. S. et al. (1997) Endocrinology 138:4013-4021; Weinstein, R. S. and S. C. Manolagas (2000) Am J.Med. 108:153-164; Weinstein, R. S. et al. (1997) Endocrinology 138:4013-4021; Weinstein, R. S., et al. (1999) J.Bone.Miner.Res. 14:S451(Abstr)); and even more important, can prevent bone loss in ovariectomized females (Vanderschueren, D., E. et al. (1992) Endocrinology 130:2906-2916; Tobias, J. H. et al. (1994) Am.J.Physiol.Endocrinol.Metab. 267:E853-E859; Coxam, V. et al. (1996) Bone 19:107-114, Lea, C. K. and A. M. Flanagan. (1999) J.Endrocrinol. 160:111-117). To date, there is no explanation for why bone cells express only small levels of the ER and AR compared to reproductive tissues, or for the lack of gender distribution of ER and AR between male and female bone cells, or the puzzling efficacy of either class of sex steroids in females and males.

This state of misunderstanding regarding the sex-non-specific efficacy of either class of sex steroids in females and males and the role of sex steroid receptors in these effects has been further confused by the ambiguous skeletal phenotype of knockouts of the ERα or ERβ in mice, designated αERKO and βERKO, respectively (Couse, J. F. and K. S. Korach (1999) Endocr.Rev. 20:358-417). αERKO or both ERα and ERβ (Double-ER-Knockout mice, DERKO) exhibit a weak bone phenotype characterized by a decrease in the length and diameter of the femur in both females and males, but only a purported minor and localized decrease of bone mineral density in the femur. ERβ knockout (βERKO) mice on the other hand exhibit longer femora and increased cortical periosteal circumference in females, but no change in males (Windahl, S. H. et al (1999) J Clin.Invest. 104:895-901) Nonetheless, volumetric pQCT or histomorphometry failed to reveal any changes in these knockout mice as compared to wild type controls, indicating that the purported changes in bone mineral density (by DEXA) are most likely the result of smaller bones. Importantly, the decreased length of the femur in the αERKO and DERKO mice is diametrically opposite to the increased length of long bones of patients with hypogonadism (including the aforementioned cases (Smith, E. P., et al. (1994) N.Engl.J.Med. 331:1056-1061; Carani, C. et al. (1997) N.Engl.J Med. 337:91-95; Bilezikian, J. P. et al. (1998) N.Engl.J Med. 339:599-603), which results from delayed epiphyseal closure. Therefore, the minor phenotypic changes in the ERKO mice, if any, may be relevant to loss of estrogenic effects on skeletal growth, but they are not informative in respect to the effects of estrogen on pubertal or adult bone remodeling. An equivocal bone phenotype has been also observed in aromatase-deficient (ArKO) mice (Oz, O. K. et al. (2000) J Bone Miner.Res 15:507-514). ArKO males, but not females, have decreased long bone growth. On the other hand, there is increased turnover in the females, but decreased turnover in the males. This result strongly argues against the contention that estrogen is the bone active sex steroid in either sex. Importantly, wall thickness, a surrogate for osteoblast number or survival, in one of the two male patients (that a bone biopsy was performed) as well as in male and female mice with aromatase deficiency is estrogen-dependent and can be corrected following treatment with estrogen (Vanderschueren, D. et al. (1998) Bone 23:391-394). On the other hand, turnover, as reflected by changes in osteoclastogenesis and osteoblastogenesis and thereby cell number, are estrogen-dependent in the females (exhibiting the expected increase secondary to estrogen deficiency), but androgen-dependent in the male (exhibiting the expected suppression secondary to androgen excess). Likewise, androgen-resistant male rats and mice (a model of testicular feminization, Tfm) have decreased bone length but normal trabecular bone volume, in spite of the fact that they have higher estrogen concentrations (resulting from aromatization of their nonfunctional androgens) (Young, C. Y. et al. (1989) Endocrinology 124:771-775; Gaspar, M. L. et al. (1991) Proc.Natl.Acad.Sci.U.S.A. 88:8606-8610; Murphy, L. and P. J. O'Shaughnessy (1991) J.Endocrinol. 131:443-449; Uesugi, Y. et al. (1992) Anat.Rec. 234:541-548; Vanderschueren, D. et al. (1993) J.Bone Miner.Res. 8:801-809).

WO 00/20007 filed by the Board of Trustees for the University of Arkansas discloses a method for increasing bone mass in a host by administering a compound that binds to the estrogen α or β receptor (and in one aspect of that embodiment, wherein the compound is not an estrogen) or the androgen receptor (and in one aspect of that embodiment, wherein the compound is not an androgen) with an association constant of at least 10⁸ M⁻¹ and induces gene transcriptional activity at a level of no greater than 5% that of 17β-estradiol while inducing an increase in extracellular signal regulated kinase (ERK) activity. WO 00/20007 emphasizes that one method to preserve the ability of an estrogen or androgen to increase ERK activity without increasing transcriptional activity of the estrogen or androgen is to attach a substituent or moiety to the estrogen or androgen to prevent it from penetrating the cell, and thus traveling to the nucleus to activate gene activity.

McDonnell and co-workers have isolated several classes of small (11-19 amino acids) peptides that bind distinct sites of the ER, and can selectively block ERα but not ERβ-mediated transcription, and vice versa, when tested on a consensus ERE (Estrogen Response Element) (Norris, J. D. et al. (1999) Science 285:744-746; Chang, C. et al. (1999) Mol.Cell Biol. 19:8226-8239).

### Screening Methods

Several methods for screening for agents that act as or through steroid receptors are known in the art. For example, U.S. Pat. No. 5,071,773 to Evans et al. discloses and claims methods for determining whether a protein suspected of being a hormone receptor stimulates transcription and also for determining whether a compound is a functional ligand for receptor proteins.

U.S. Pat. No. 5,298,429 also to Evans et al. discloses a bioassay for determining whether compounds are functional ligands of hormone receptor proteins by assaying the effect of a compound on transcription in cultured cells expressing non-endogenous DNA encoding a hormone receptor protein or a variant thereof and DNA encoding an operative hormone response element linked to a reporter gene.

General bioassays include U.S. Pat. No. 5,877,007 to Housey which discloses a bioassay for compounds that activate a protein expressed in cells at a higher level than control cells such that the compound-activated protein induces a phenotypic change in the cells.

WO 00/37681 in the name of American Home Products Corp. discloses bioassays for identifying receptor modulators specific for either ERα or ERβ. The disclosed assays use two sets of cells in which the first set contains DNA encoding a functional ERα and a responsive reporter gene construct, and the second set of cells contains and ERE reporter construct and ERβ. Compounds can be screened for selective activation or inhibition of transcription in cells expressing ERα or ERβ.

U.S. Pat. No. 5,643,720 discloses methods for screening for ligands that bind to the retinoid receptor to form a complex which binds AP-1 and prevents DNA binding. US Pat. No. 5,702,914 discloses bioassays for determining whether a compound is a functional ligand of a retinoid receptor by assaying for transcription of a reporter gene construct in genetically modified cells.

U.S. Pat. No. 5,863,733 discloses methods for determining whether a chemical specifically modulates the transcriptional activity of a gene of interest using a reporter gene construct in genetically altered cells.

U.S. Pat. No. 5,563,036 discloses methods for screening for compounds that inhibit the binding of transcription factors to nucleic acids by detecting the presence or absence of labeled transcription factors on a solid substrate when treated with a test compound.

U.S. Pat. No. 5,854,004 discloses methods for screening for compounds that modulate a receptor-dependent signal transduction pathway by monitoring the transcription of a reporter gene in cells genetically modified to contain a reporter gene and a DNA regulatory sequence responsive to changes in concentration of inositol- 1, 4,5-triphosphate (IP₃) and diacylglycerol.

U.S. Pat. No. 5,639,597 top Lauffer et al. discloses a cell-free assay to determine whether a compound is a ligand of a receptor.

U.S. Pat. No. 6,060,238 to Dixit discloses a method for screening for agents that inhibit apoptosis as compared with cowpox cytokine response modified A.

U.S. Patent No. 5,506,102 filed by McDonnell et al., describes methods of using the A form of the progesterone receptor to screen for antagonists of steroid intracellular-mediated transcription.

U.S. Patent No. 5,834,213 discloses a tissue culture screening system to monitor a transcriptional response treated by a chemical signal interacting with a plasma membrane receptor. The issue culture screening system includes a cell line containing a membrane receptor, a target gene and a specific receptor selected from the group consisting of a steroid receptor, a vitamin receptor and an orphan receptor.

WO 99/54728 filed by Novalon Pharmaceutical Corporation describes a method to predict whether a test compound will modulate the biological activity of a receptor in a multicelular organism on the basis of its interaction with peptide or nucleic acid ligands that modify the conformation of the receptor. The data is compared to the fingerprints for reference compounds with known biological activities.

### Other References of Interest

U.S. Pat. Nos. 5,362,720; 5,545,634; 5,846,960 and 5,567,695 to Labrie disclose a method for the treatment or prevention of breast and endometrial cancer, osteoporosis and endometriosis in susceptible warm-blooded animals comprising administering a low dose of a progestin or other steroid derivative having androgenic activity and low masculinizing activity. In a primary embodiment, the method involves activating the androgen receptor by administering at least one androgenic steroid having a Ki value of less than 2×10⁻⁸ for the androgen receptor, an androgen receptor-mediated inhibitory effect on the growth of human breast cancer ZR-75-1 cells which reaches half-maximal value at a concentration below 3.0 nanomoles per liter, and no visible masculinizing activity at blood serum concentrations below 50 nM, wherein every such androgenic steroid is administered at a dosage sufficiently low to maintain a cumulative serum concentration of below 50 nanomoles per liter.

U.S. Patent No. 5,843,934 to Simpkins claims that an estrogen having insubstantial sex-related activity can be administered to a patient to retard the adverse effects of osteoporosis in a male or female. The '934 patent does not address how to select a compound to increase bone mass, but instead teaches how to retard the effect of bone loss. In a preferred embodiment, the estrogen used is 17α-estradiol. The '934 patent states that the inventors do not understand the mechanism of how 17α-estradiol exerts a cytoprotective effect, but suggests that it exerts a direct protective effect (assumably as opposed to through a receptor action). One example given of a direct protective effect is the modulation of the regulation of glucose (see columns 7 and 8 of the '934 patent).

WO 98/22113 filed by the University of Florida Research Foundation, Inc. discloses methods to utilize an α or β-isomer of an estrogen compound to confer cytoprotection on a population of cells associated with an ischemic event.

U.S. Pat. No. 5,859,001 discloses the use of non-estrogen compounds having a terminal phenol group in a four-ring cyclopentanophenanthrene compound structure to confer neuroprotection to cells.

WO 98/31381 filed by the University of Florida Research Foundation, Inc. discloses a method for enhancing the cytoprotective effect of polycyclic phenolic compounds on a population of cells that involves the steps of administering a combination of polycyclic phenolic compounds and anti-oxidants to achieve an enhanced effect. One disclosed combination is glutathione and estrogen.

WO 99/61044 filed by the Board of Trustees for the University of Arkansas discloses the use of noggin or antagonists of noggin as therapeutic agents in the regulation of osteoblastogenesis and osteoclastogenesis to control bone remodeling. WO 22/20625 also to the Board of Trustees of the University of Arkansas discloses that glucocorticoid-induced bone disease is due to changes in the birth and death rate of bone cells and that glucocorticoid administration increases apoptosis of mature osteoblasts and osteocytes and decreases bone formation rate and bone mineral density.

Furthermore, WO 00/19823 to the Board of Trustees of the University of Arkansas discloses that human parathyroid hormone 1-34 [hPTH(I-34)] exerts anti-apoptotic effects on osteoblasts when administered in an intermittent fashion to mice in vivo. That same application also provides evidence that bovine PTH(1-34) [bPTH(I-34)] prevents g lucocorticoid -induced apoptosis of osteoblastic and osteocytic cells in vitro.

WO 00/20625 also to the Board of Trustees of the University of Arkansas discloses that glucocorticoid administration increases apoptosis of mature osteoblasts and osteocytes and decreases bone formation rate and bone mineral density accompanied by defective osteoblastogenesis and osteoclastogenesis in the bone marrow.

From the above discussion, it is apparent that in spite of considerable progress toward an understanding of the steroid signaling, there is a lack of understanding of how steroids mediate nongenotropic and genotropic effects.

It is an object of the present invention to induce selective steroidal effects.

It is also an object of the present invention to provide a method to treat steroid and or steroid receptor related disorders or diseases.

It is yet another object of the present invention to provide a transgenic animal to screen for compounds that can induce a selective steroidal response.

It is yet another object of the present invention to provide a method to select compounds that are useful to induce a selective steroidal response.

### SUMMARY OF THE INVENTION

This invention concerns the fundamental discovery of the understanding of the mechanism of action of steroidal nongenotropic effects and their relation to steroidal genotropic effects. It has been discovered that (i) steroidal nongenotropic effects and genotropic effects can be mediated by the same steroid receptor; (ii) both effects are ligand-induced; (iii) the nongenotropic effect occurs due to a ligand interaction in the ligand binding domain of the steroid receptor, which can be fast and loose fitting; (iv) the genotropic effect occurs due to a ligand interaction with the ligand-binding domain but requires the DNA-binding domain of the steroid receptor, which is typically a result of a slower, stronger interaction; and (v) the nongenotropic effect of a ligand interaction can be dissociated from the genotropic effect of a ligand interaction, in such a manner to effect a selective response.

Based on this pioneering discovery, a method is provided of dissociating a steroidal nongenotropic effect from the steroidal genotropic effect of a steroid receptor. In one embodiment, the invention includes a method for selectively inducing a steroidal nongenotropic effect, that includes contacting the receptor with a compound that sufficiently interacts with the ligand binding domain of the receptor in a manner that causes the receptor to mediate a nongenotropic effect, while not activating the DNA-binding domain such that a significant genotropic effect is not produced. In an alternative embodiment, a method is provided for selectively inducing a steroidal genotropic effect, that includes contacting the receptor with a compound that sufficiently activates the DNA-binding domain of the receptor in a manner that causes the receptor to mediate a genotropic effect, while not causing a significant nongenotropic effect. It is considered that a ligand does not induce a “significant” or “substantial” effect if the effect is less than 10% that of the natural steroidal effect, and in a preferred embodiment, less than 5, 1, or 0.1 percent that of the natural steroidal effect.

Exemplary forms of nongentropic activities include but are not limited to activation of intracellular second messenger systems, signal transduction pathways, protein kinase signal transduction pathways, MAP kinase signal transduction pathways, Src/Shc/ERK signal transduction pathways; regulation of intracellular calcium concentration; secretion; changes in cellular morphology; cell motility; cytoskeletal rearrangements; and apoptosis.

In another aspect of the invention, a selectivity inducing compound (including but not limited to a peptide or protein or a ligand antagonist) can be administered that competes with an endogenous or exogenous ligand of a steroid receptor for a binding site on the receptor as a means to mask the activity of that ligand-binding interaction. As one example, a peptide or ligand antagonist can be administered that binds with the ligand binding domain in a way that prevents a ligand from inducing a nongenotropic effect. In an alternative embodiment, a peptide or ligand antagonist can be administered that binds with the ligand-binding domain of the receptor in a way that prevents a ligand from inducing a genotropic effect. The selectivity-inducing compound can inhibit the transcriptional activity of the steroid receptor directly by interfering with DNA-receptor interactions or indirectly by preventing the receptor from forming a protein-protein interaction with other transcriptional regulators. Additionally, the selectivity inducing compound can exist as a complex having at least two components such that when the components combine, the complex inactivates the genotropic activity and activates the nongentropic activity of a steroid receptor.

In a still another aspect, a method for inducing a selective effect of a steroid receptor is provided that includes administering an effective amount of a compound that inactivates the DNA binding region of the receptor by inducing a conformational or configurational change in the receptor. In yet another aspect of the invention, a method for inducing a selective effect of a steroid receptor is provided that includes administering an effective amount of a compound that inactivates functions mediated only by the ligand binding region of the receptor by inducing a conformational or configurational change in the-protein.

In an alternative embodiment, a method is provided for screening compounds that are capable of inducing a steroidal nongenotropic effect without substantially inducing a steroidal genotropic effect or are capable of inducing a steroidal genotropic effect without substantially inducing a steroidal nongenotropic effect. The method can be carried out in a number of ways, including:

(1) assessing the ability of the compound to appropriately bind to the ligand-binding domain of the steroid receptor without substantially activating the DNA-binding domain of the steroid receptor or to appropriately activate the DNA-binding domain of the steroid receptor without substantially activating functions that are mediated only by the ligand-binding domain of the steroid receptor; and then

(2) measuring the biological activity of the test compound to assess its ability to induce a target nongenotropic effect without substantially inducing a genotropic effect or its ability to induce a target genotropic effect without inducing a substantial nongenotropic effect.

The first step can be accomplished via molecular modeling, preferably with the aid of a computer. Alternatively, modified forms of the receptor consisting only of the ligand-binding domain and lacking a functional DNA binding domain can be used to determine whether the test compound activates the ligand binding domain or DNA binding domain. The modified form can be expressed, for example, in a transformed cell. Cells cultured in vitro can be genetically altered to express a steroid receptor or a genetic variant thereof. Genotropic activity can be quantified using known techniques such as measuring the amount of total mRNA generated in response to a test compound or the mRNA specific for one or more steroid regulated genes. Screening can be done in vivo or in vitro.

Alternatively, steps 1 and 2 can be combined by simply assaying for compounds that selectively induce either a nongenotropic or genotropic effect without substantially inducing the other effect in an in vitro cell-based assay.

This invention can be used to selectively effect a response in any of the family of steroid receptors, including the α or β form of the estrogen receptors (ERα and ERβ), the estrogen receptor related receptor-1 and receptor-2 (ERR-1 and ERR-2), the androgen receptor (AR), the progesterone receptor (PR), retinoic acid receptor (and related orphan receptors ROR-α, RORβ, and ROR-γ); glucocorticoid receptor (GR), mineralcorticoid receptor (MR), Vitamin D receptor, neuroactive receptor, farnesoid X receptor (FXR), liver X receptor (LXRα and LXRβ), thyroid hormone receptors A and B (which bind triiodothyronine (T3) and thyroxine (T4)), COUP-TF, ecdysone, the PPAR family (peroxisome proliferator activated protein, including PPARα, PPARγ, and PPARδ), pregnane X (“PXR”), bile acid binding family, and chimeric and orphan receptors thereof.

In another embodiment of the invention, it has been discovered that steroids can induce a nongenotropic effect via an interaction with a steroid receptor other than its typical binding receptor. In one nonlimiting illustration of this aspect of the invention, it has been discovered that the estrogen receptors (ERα and ERβ) and the androgen receptor (AR) can transmit anti-apoptotic signals to osteoblasts and osteocytes with similar efficiency irrespective of whether the binding ligand is an estrogen or an androgen. Further, it has been discovered that the nongentropic receptor-dependent anti-apoptotic mechanism of action of estrogens and androgens, which in the case of osteoblasts/osteocytes involves activation of extracellular signal regulated kinases (ERKs) are mechanistically dissociable from the transcriptional activity of the receptors.

The fact that a steroid can use a previously considered unrelated steroid receptor to induce a nongenotropic effect now explains the anomaly described in the Background of the Invention that nongenotropic steroid effects occur in cells or tissues devoid of the respective classic receptor. In such a case, the steroid is using an unrelated steroid receptor to mediate that nongenotropic effect.

As one illustration, by testing human estrogen receptor clones that have mutations in various domains of the protein, the area of the receptor responsible for nongenotropic effects (for example, an anti-apoptotic effect) has been located. The term anti-apoptotic domain as used herein refers to that location on the protein that mediates an anti-apoptotic effect on osteoblasts and osteocytes when bound to an anti-apoptosis-inducing ligand, such as an estrogen or an androgen. It has been discovered that the anti-apoptotic domain of the estrogen receptor is in the ligand binding region (domain E). It has specifically been found that when a synthetic peptide (αII) binds to the ligand bound-estrogen receptor at domain E, which is known to effectively blockade the transcriptional activity of the estrogen receptor, there is no effect on the subsequent anti-apoptotic effect of that ligand bound estrogen receptor.

Using the ERα as a prototype, it was next investigated whether the anti-apoptotic activity of the receptors for sex steroids requires the same or different domains than those required for transcriptional activity (FIG. 3A). To do this, the effects of several mutants of the ERα on prevention of apoptosis were compared. These mutants have been shown to produce the expected proteins when transiently transfected into human cells (Schodin et al., 1995; Kraus et al., 1997; Ekena et al., 1996). It was shown that the mutants comprising the dimerization (D) and the ligand-binding domain (E) or only the E domain also produced the expected proteins and lacked transcriptional activity.

Like ERα, mutant ΔA/B which lacks the entire N-terminal transcription activation function (AF-1) domain, or a mutant in which serines were substituted with alanine (S104,106,118A), or mutant AF, which lacks the F domain, or mutant ΔDBD which lacks most of the DNA binding domain (amino acids 185-251), were able to convey the anti-apoptotic signal of the E₂-bound ER. Yet, all these mutants exhibit reduced or no ERE-mediated gene transcriptional activity. Le Goff, et al, J. Biol. Chem. 269, 4458-4466; mcInemey, et al., J. Biol. Chem., 271, 24172-24178; Katzenellebogen. More striking, the ability of the ligand to convey the anti-apoptotic signal was preserved in a deletion mutant which is missing the entire AF-1 and DNA binding domain (DE); as well as in a deletion mutant consisting solely of the AF-2/ligand binding domain (E). In contrast, the ER mutant L525A which lacks hormone binding ability and S554fs which is deficient in the C-terminal transcription activation function (AF-2), lost the ability to convey both the anti-apoptotic signal and the transcriptional activity of the protein. It was next determined whether the anti-apoptotic activity of the E domain was dependent on its localization to a particular subcellular compartment (FIG. 3B). This domain was targeted to the plasma membrane or to the nucleus of HeLa cells, by fusing it to enhanced cyan fluorescent protein containing plasma membrane or nuclear localization sequences, respectively. As revealed by epifluorescence microscopy, the non-targeted ERα and the non-targeted E exhibited similar distribution of cyan fluorescence in the nucleus and cytoplasm, indicating that elimination of all but the ligand binding domain of the ER does not alter the subcellular distribution. However, incorporation of the appropriate targeting sequences accomplished the expected subcellular localization of the membrane or the nuclear ECFP fusion proteins, respectively, as can be seen from the distribution of cyan and red fluorescence in the same cell(s). Targeting the E domain predominantly to the plasma membrane did not alter its ability to convey the ligand-induced anti-apoptotic signal, as evidenced by identical anti-apoptotic activity of this fusion protein to that of the E domain or the full length ERα. But in sharp contrast, targeting the E domain exclusively to the cell nucleus resulted in complete loss of its anti-apoptotic activity.

### BRIEF DESCRIPTION OF THE DRAWINGS

The Figures provided herein illustrate embodiments of the invention and are not intended to limit the scope of the invention.

FIG. 1 is a series of graphs demonstrating the control of osteoblast and osteocyte apoptosis by sex steroids in vivo and in vitro. A. Prevalence of osteoblast and osteocyte apoptosis following ovariectomy or orchidectomy in mice. Bars are means±SD of 4-5 animals per group. *p<0.05. B. Calvarial osteoblasts were treated for 1 hour with 10⁻¹¹ M to 10⁻⁶ M of E₂ or DHT followed by 6 hours treatment with the pro-apoptotic agent etoposide (50 μM). The percentage of dead cells was quantified by trypan blue staining. Etoposide-induced apoptosis in the absence of steroids was set at 100%. Bars indicate means±SD of triplicate determinations, *p<0.05 for E₂- or DHT-treated cultures versus vehicle using separate one-way ANOVAs for each treatment. C and D. Calvaria-derived osteoblasts or MLO-Y4 osteocytic cells were treated with 10⁻⁸ M E₂ or DHT, and apoptosis was induced by addition of etoposide, dexamethasone (10⁻⁷ M), or TNFA (1 nM). The percentage of dead cells was quantified as in B. Results were analyzed by two-way ANOVA. *p<0.05 when comparing pretreatments within each agent group and each agent versus vehicle for a given pretreatment. E. OB-6 osteoblastic cells incorporated into collagen gels were treated with different concentrations of E₂ or DHT for 24 hours. A total of 200 cells were evaluated in randomly selected fields. In vehicle controls, 32.3+2.5% of the cells were apoptotic. *p<0.05 for E₂- or DHT-treated cultures versus vehicle using separate one way ANOVAs for each treatment.

FIG. 2 is a series of graphs showing that the anti-apoptotic effects of E₂ or DHT are inhibited by both ER or AR antagonists and can be mediated via either the ER (α or β) or the AR. A. Calvarial osteoblasts were pre-treated with the ER antagonist ICI 182,780 (10⁻⁷ M) or with the AR antagonist flutamide (10⁷ M) for 30 min, followed by incubation with 10⁻⁸ M E2 or DHT for 1 hour. Subsequently, etoposide was added and apoptotic cells were quantified after 6 hours as in FIG. 1B. Results are expressed as percentage of etoposide-induced apoptosis in the absence of steroids which was set at 100%. Bars indicate means±SD of triplicate determinations, *p<0.05 versus vehicle, by ANOVA. B. HeLa cells were transiently transfected with either ERα, ERβ, AR, VDR, or RXR expression vectors or with the empty vector (ev), along with a Nuc-EGFP expression vector. Sixteen hours after transfection, cells were treated with the indicated concentrations of E₂, DHT, or 1,25-dihydroxy-Vitamin D₃ (1,25D₃) for 1 hour followed by 6 hours treatment with etoposide. Apoptotic cells were identified by examining the nuclear morphology of 200-500 transfected (fluorescent) cells. Etoposide-induced apoptosis in cells cultured in the absence of steroids (−) was set at 100%. Results are expressed as percentage of etoposide-induced apoptosis (mean±SD). * p<0.05 versus cells treated with etoposide in the absence of steroids by ANOVA.

FIG. 3 shows the localization of the anti-apoptotic activity of the ERα to the E (ligand binding) domain and elimination by nuclear targeting. A. HeLa cells were co-transfected with expression vectors for Nuc-EGFP and various ERα mutants, and then treated for 1 hour with 10⁻⁸ M E₂ followed by 6 hours treatment with etoposide. B. The full length ERα was fused to the non-targeted ECFP (ERα-ECFP). The E domain of the ERα was fused to either non-targeted ECFP (E-ECFP), membrane targeted ECFP (E-Mem-ECFP) or nuclear targeted ECFP (E-Nuc-ECFP). Each of these fusion proteins was expressed in HeLa cells along with a nuclear targeted red fluorescent protein (Nuc-ERFP) and their subcellular distribution was analyzed by epifluorescence microscopy. Left and middle panels depict photomicrographs of the same cell(s) obtained with either cyan or red filter sets, respectively; bar=15 μm. Nuc-ERFP was used in this experiment to quantify apoptosis because its emission can be readily distinguished from that of the cyan fusion proteins. Transfected cells were treated for 1 hour with 10⁻⁸ M E₂ or vehicle followed by 6 hours treatment with etoposide. Apoptosis in both 3A and 3B was quantified as in FIG. 2. Bars indicate means±SD of triplicate determinations. *p<0.05 versus cells not treated with etoposide by ANOVA.

FIG. 4 shows that the anti-apoptotic activity of the ER and AR is mediated via a Src/Shc/ERK signaling pathway. A. MLO-Y4 cells were incubated for 2, 5, or 15 min with E₂ or DHT or for 25 min with PD98059 (50 μM) or PPI (10 μM) followed by 5 min with E₂ or DHT. Cell lysates were obtained and proteins were analyzed by Western blot analysis using anti-ERK1/2 or anti-phospho-ERK1/2 (p-ERK1/2) antibodies. For the determination of apoptosis, cells were incubated with vehicle, PD98059 or PP1 30 min prior to the addition of E₂ or DHT. After 30 min, etoposide was added and apoptosis was assayed 6 hours later as in FIG. 1B. B. The effect of E₂ or DHT on apoptosis was compared in embryonic fibroblasts from Src+/+ versus Src−/− mice, as described in FIG. 1B. RT-PCR was performed as described in the experimental procedures. C-E. HeLa cells were transfected with Nuc-EGFP and with expression vectors encoding the full length ERα or its E domain, or the AR alone (C, left panel) or together with a dn MEK (C, right panel); a Src mutant lacking the kinase activity (Src K⁻), a Src mutant lacking the SH2 domain (Src ΔSH2), or a Src mutant lacking the SH3 domain (Src ΔSH3) (D); or with ERα together with wt Shc, or the dn Shc mutants Y239F/Y240F/Y317F (Shc FFF), Y317F (Shc YYF) or Y239F/Y240F (Shc FFY) (E). Bars represent means±SD of triplicate determinations. *p<0.05 versus cells cultured without steroids, by ANOVA.

FIG. 5 demonstrates the dissociation of ERα -mediated transcriptional versus anti-apoptotic actions and ERα -mediated versus ERβ-mediated effects using peptides. A. HeLa cells were transiently transfected with the ERα expression vector alone or together with a vector carrying a fusion of the α-II peptide with the GAL4 DNA binding domain driven by the SV40 promoter. In addition, cells were transfected with ERE-luc or IL-6-luc for the transcription studies; or with EGFP-nuc for the apoptosis experiments. Bars represent the mean±SD of triplicate determinations and indicate fold-stimulation of ERE-luc, or fold-inhibition of IL-6-luc, or % of transfected cells with apoptotic features in vehicle (E₂−) or 10⁻⁸ M E₂ (E₂+). * p<0.05 versus vehicle treated cells, by ANOVA. B. HeLa cells were transfected with ERα or ERβ alone or together with expression vectors carrying 2X293 or 2XF6 peptides. Bars represent mean±SD of triplicate determinations of the activity (ERE-luc, IL-6-luc, or anti-apoptosis) in response to E₂ (10⁻⁸ M) in cells transfected with the indicated receptor and peptide versus cells transfected with the receptor alone. E₂-induced activity in cells carrying the receptor, but not the peptide, was designated as 100%. * p<0.05 versus cells transfected with the receptor alone, by ANOVA. C. HeLa cells were transfected with a vector carrying the GRIP peptide alone or together with ERα. Bars represent the mean±SD of triplicate determinations. * p<0.05 versus vehicle treated cells, by ANOVA.

FIG. 6 demonstrates the dissociation of nongenotropic from genotropic activity of the ER with synthetic ligands. A. For C3-mediated transcription, HeLa cells were transfected with C3-luc and ERα, and were treated with the indicated concentrations of an estren or a pyrazole. Bars represent the mean±SD of the relative luciferase units (RLU) normalized for β-galactosidase activity. p<0.05 versus cells transfected with empty vector (ev), by ANOVA. The anti-apoptotic activity of the estren and the pyrazole was evaluated in calvaria-derived osteoblasts as described in FIG. 1B. * p<0.05 versus cells not treated with etoposide by ANOVA. B. MLO-Y4 cells were incubated for 5, min with the indicated concentrations of the different ligands and ERK phosphorylation was assessed as in FIG. 4A.

FIG. 7 shows a proposed model for ligand-induced dissociation of anti-apoptotic from classical genotropic activity of sex steroid receptors. The three cartoons depict conformational states of the receptor protein prior to and following interaction with the ligands, that are required to effect either the genotropic or the anti-apoptotic responses. The inactive unligated receptor is depicted in the middle in gray. The change in conformation induced by interaction with a ligand that preferentially triggers transcriptional activity (e.g., the pyrazole of FIG. 6) is depicted in the right in blue. The change in conformation induced by interaction with a ligand that preferentially triggers the anti-apoptotic activity of the receptor (e.g., the estren of FIG. 6) is depicted in the left in magenta. Green circle and green diamond represent the two ligands; please note the perfect and imperfect fit within the binding pocket, respectively. Association (kₐ) and dissociation (k_{d}) rates of the two different types of ligands for the receptor are depicted by different width and length arrows. Ligands such as E₂ will of course induce both conformations. Although in the anti-apoptotic model a direct contact between the receptor and Src is shown, it is possible that adapter protein(s) may bridge the interaction between the two molecules. Corresponding energy levels of the receptor protein in the inactive unligated state (broken line), progressing either to the genotropic conformation (blue line) or the anti-apoptotic conformation (magenta line) are shown in the bottom. The discontinuous vertical axis in the energy diagram is meant to indicate a larger than scale difference in energy requirements between the two activities.

FIG. 8 is a diagram of two transgene constructs that are co-introduced into the same mouse for Dox-induced expression of the αII and 293 peptides.

FIG. 9A is a diagram illustrating the generation and functional characterization of the Dox-inducible system for osteoblast-specific expression of Cre recombinase. Activation of the ROSA26 locus by Cre under the control of the OG-2 promoter. “SA” indicates a splice acceptor site of the original ROSA26 locus. FIG. 9B is gel showing Dox-induced activation of Cre expression in vitro. Ob-6 cells were transiently transfected with the Tet-OG2-Cre constructs and were then treated with 0.5mg/ml Dox or vehicle for 24 hours. Cells were harvested and Cre expression was assessed by RT-PCR analysis. Lane 1 contains size markers. Lane 2 represents cells treated with Empty Vehicle plus Dox. Lane 3 represents cells containing OG-2-rtA-Cre without Dox. Lane 4 represents cells containing OG-2-rtA-Cre with Dox. Lane 4 contains the OG-2rtA-Cre plasmid induced with Dox.

FIG. 10 is a diagram illustrating the targeting of floxed exon 3 into a 86 Kb genotropic locus containing exons 3 and 4 of the mouse ERα gene.

### DETAILED DESCRIPTION OF THE INVENTION

This invention concerns the fundamental discovery of the understanding of the mechanism of action of steroidal nongenotropic effects and their relation to steroidal genotropic effects. It has been discovered that (i) steroidal nongenotropic effects and genotropic effects can be mediated by the same steroid receptor; (ii) both effects are ligand-induced; (iii) the nongenotropic effect occurs due to a ligand-induced activation of the ligand binding domain, which can be fast and loose fitting; (iv) the genotropic effect occurs due to a ligand-induced activation of the DNA-binding domain of the steroid receptor, which is typically a result of a slower, stronger interaction; and (v) the nongenotropic effect of a ligand interaction can be dissociated from the genotropic effect of a ligand interaction, in such a manner to effect a selective response.

Based on this pioneering discovery, a method is provided of dissociating a steroidal nongenotropic effect from the steroidal genotropic effect of a steroid receptor. In one embodiment, the invention includes a method for selectively inducing a steroidal nongenotropic effect, that includes contacting the receptor with a compound that sufficiently interacts with the ligand binding domain of the receptor in a manner that causes the receptor to mediate a nongenotropic effect, while not activating the DNA-binding domain in a manner that induces a genotropic effect. In an alternative embodiment, a method is provided for selectively inducing a steroidal genotropic effect, that includes contacting the receptor with a compound that sufficiently activates the DNA-binding domain of the receptor in a manner that causes the receptor to mediate a genotropic effect, while not activating the ligand-binding domain in a manner that induces a nongenotropic effect. In one aspect of all of the inventions disclosed herein, the receptor is not an estrogen receptor, or alternatively, is not a progesterone receptor.

I. Definitions

An estrogen compound, as used herein, refers to a four ring steroidal compound which possesses the biological activity of an estrus-producing hormone, or its conjugated and esterified derivative, or a derivative thereof of same chemical composition and structure but which does not possess the biological activity of the active form because it exhibits a different stereochemistry from the active form. Nonlimiting examples of estrogens include broparestrol, chlorotrianisene, dienoestrol, epimestrol, equilin, estrapronicate, estropipate, ethinylestradiol, fosfestrol, hydroxyesetrone, mestranol, estradiol, estriol, conjugated and esterified estrogens, estrone, polyestradiol, promestriene, quinestradol, quinestrol, stilbestrol, and zeranol.

An androgen compound, as used herein, refers to a four ring steroidal compound which can be produced in the testis or adrenal cortex, or is a synthetic hormone, which acts to regulate masculine secondary sexual characteristics, or a derivative thereof of same chemical composition and structure but which does not possess the biological activity of the active form because it exhibits a different stereochemistry from the active form. Nonlimiting examples include boldenone, clostebol, danazol, drosstanolone, epitiostanol, ethylestrenol, fluoxymesterone, formebolone, furazabol, mepitiostane, mesterolone, methandienone, methenolone, methyltestosterone, nandrolone, norethandrolone, oxabolone, oxymetholone, prasterone, quinbolone, staolone, stanozolol, testosterone, and trenbolone.

As known, estrogens and androgens have chiral carbons, and thus can exist in a number of stereochemical configurations. Typically, for example, the 17β hydroxy estrogens have biological activity while the 17α hydroxy estrogens have very little effect on sexual characteristics (and induce little hormone-like gene transcriptional activation). For the purpose of this specification, any stereochemical configuration, including either the biologically active or the biologically inactive or less active structure, can be used, as long as the compound satisfies the specifically itemized criteria of the invention.

The catalogue entitled “Steroids” from Steraloids Inc., Wilton H. H., provides a list of over 3000 steroids, with numerous estrogen and androgen derivatives. The catalog can be obtained by contacting the company and is also currently available on the internet at http://www.steraloids.com. One can select and purchase compounds from this library, which are all commercially available and thus easy to obtain and evaluate, for use in this invention.

The term “steroid” as used herein refers to any ligand that will bind to one of the traditionally referred to “nuclear hormone receptor family of proteins.” Based on the discovery herein, it is now known that these receptors are not necessarily nuclear, and in fact can induce nongenotropic effects when located outside the nucleus or on the membrane, and perhaps in the nucleus as well. The names of the individual members of the family of the nuclear receptor family is published in Cell, 97:161-163, 1999, and reproduced in the Table below. This article was authored by the Nuclear Receptors Nomenclature Committee. It is now known that there are at least sixty members of the protein family in humans as shown by analysis of the human genome and proteome, and published in Nature 409:860-921, 2001. This article was authored by the International Human Genome Sequencing Consortium. In one embodiment, the receptor has a zinc finger and a ligand-binding domain, as mentioned in the

TABLE 1
A Proposed Nomenclature for Nuclear Receptors

| Subfamily and | | | |
|---|---|---|---|
| Group | NR/Gene | Trivial Names | Accession Number |
| 1A | NR1A1 | TRα, c-erbA-1, THRA | M24748 |
| | NR1A2 | TRβ, c-erbA-2, THRB | X04707 |
| 1B | NR1B1 | RARα | X06538 |
| | NR1B2 | RARβ, HAP | Y00291 |
| | NR1B3 | RARγ, RARD | M57707 |
| 1C | NR1C1 | PPARα | L02932 |
| | NR1C2 | PPARβ, NUC1, PPARδ, FAAR | L07592 |
| | NR1C3 | PPARγ | L40904 |
| 1D | NR1D1 | REVERBα, EAR1, EAR1A | M24898 |
| | NR1D2 | REVERBβ, EAR1β, BD73, RVR, HZF2 | L31785 |
| | NR1D3 | E75 | X51548 |
| 1E | NR1E1 | E78, DR-78 | U01087 |
| 1F | NR1F1 | RORα, RZRα | U04897 |
| | NR1F2 | RORβ, RZRβ | Y08639 |
| | NR1F3 | RORγ, TOR | U16997 |
| | NR1F4 | HR3, DHR3, MHR3, GHR3, CNR3, | M90806 |
| | | CHR3 | U13075 |
| 1G | NR1G1 | CNR14 | U13074 |
| 1H | NR1H1 | ECR | M74078 |
| | NR1H2 | UR, OR-1, NER1, RIP15, LXRβ | U07132 |
| | NR1H3 | RLD1, LXR, LXRα | U22662 |
| | NR1H4 | FXR, RIP14, HRR1 | U09416 |
| 1I | NR1I1 | VDR | J03258 |
| | NR1I2 | ONR1, PXR, SXR, BXR | X75163 |
| | NR1I3 | MB67, CAR1, CARα | Z30425 |
| | NR1I4 | CAR2, CARβ | AF00932 |
| 1J | NR1J1 | DHR96 | U36792 |
| 1K | NR1K1 | NHR1 | U19360 |
| 2A | NR2A1 | HNF4 | X76930 |
| | NR2A2 | HNF4G | Z49826 |
| | NR2A3 | HNF4B | Z49827 |
| | NR2A4 | DHNF4, HNF4D | U70874 |
| 2B | NR2B1 | RXRA | X52773 |
| | NR2B2 | RXRB, H-2RIIBP, RCoR-1 | M84820 |
| | NR2B3 | RXRG | X66225 |
| | NR2B4 | USP, Ultraspiracle, 2C1, CF1 | X52591 |
| 2C | NR2C1 | TR2, TR2-11 | M29960 |
| | NR2C2 | TR4, TAK1 | L27586 |
| 2D | NR2D1 | DHR78 | U36791 |
| 2E | NR2E1 | TLL, TLX, XTLL | S72373 |
| | NR2E2 | TLL, Tailless | M34639 |
| 2F | NR2F1 | COUP-TFI, COUPTFA, EAR3, SVP44 | X12795 |
| | NR2F2 | COUP-TFII, COUPTFB, ARP1, SVP40 | M64497 |
| | NR2F3 | SVP, COUP-TF | M28863 |
| | NR2F4 | COUP-TFIII, COUPTFG | X63092 |
| | NR2F5 | SVP46 | X70300 |
| | NR2F6 | EAR2 | X12794 |
| 3A | NR3A1 | ERα | X03635 |
| | NR3A2 | ERβ | U57439 |
| 3B | NR3B1 | ERR1, ERRα | X51416 |
| | NR3B2 | ERR2, ERRβ | X51417 |
| 3C | NR3C1 | GR | X03225 |
| | NR3C2 | MR | M16801 |
| | NR3C3 | PR | M15716 |
| | NR3C4 | AR | M20132 |
| 4A | NR4A1 | NGFIB, TR3, N10, NUR77, NAK1 | L13740 |
| | NR4A2 | NURR1, NOT, RNR1, HZF-3, TINOR | X75918 |
| | NR4A3 | NOR1, MINOR | D38530 |
| | NR4A4 | DHR38, NGFIB | U36762 |
| | | CNR8, C48D5 | U13076 |
| 5A | NR5A1 | SF1, ELP, FTZ-F1, AD4BP | D88155 |
| | NR5A2 | LRH1, xFF1rA, xFF1rB, FFLR, PHR, FTF | U93553 |
| | NR5A3 | FTZ-F1 | M63711 |
| 5B | NR5B1 | DHR39, FTZF1B | L06423 |
| 6A | NR6A1 | GCNF1, RTR | U14666 |
| 0A | NR0A1 | KNI, Knirps | X13331 |
| | NR0A2 | KNRL, Knirps related | X14153 |
| | NR0A3 | EGON, Embryonic gonad, EAGLE | X16631 |
| | NR0A4 | ODR7 | U16708 |
| | NR0A5 | Trithorax | M31617 |
| 0B | NR0B1 | DAX1, AHCH | S74720 |
| | NR0B2 | SHP | L76571 |

Note: The groups contain highly related genes with often paralogous relationship in vertebrates (e.g., RARA, RARB, and RARG). The term isoform is reserved for different gene products originating from the same gene due to alternative promoter usage or splicing, or alternative initiation of translation.

Nature article. Generally, steroid receptors have a highly variable N-terminal trans-activation domain (NT-TAD), followed by a highly conserved DNA-binding domain (DBD) consisting of two zinc fingers, a hinge region, and a moderately conserved C-terminal ligand-binding domain (CT-LBD). These regions either have already been mapped for a target steroid receptor, or are easily mapped using known techniques.

The term “bone mass” refers to the mass of bone mineral and is typically determined by Dual-Energy X-Ray Absorptiometry (DEXA).

The term “bone strength” refers to resistance to mechanical forces and can be measured by any known method, including vertebrae compression strength or three point -bending of long bones.

The term “bone quality” refers to normal collagen orientation without excessive accumulation of unmineralized bone matrix, and can be measured by any known method, including undecalcified bone histomorphometry.

The term “bone anti-resorption agent” refers to a compound that blocks bone resorption by suppressing remodeling or the activity and/or lifespan of osteoclasts.

The term “osteopenia” refers to decreased bone mass below a threshold which compromises structural integrity.

The term chimera as used herein means a strand of DNA formed by fusion of the genetic sequence of two different receptors. Steroid receptors are composed of both a DNA binding domain and a ligand binding domain. In the chimeric receptor the DNA binding domain is composed of the genetic code from one receptor and the ligand binding domain of another receptor. These two domains are fused to form a chimeric receptor. The domains can be from different species or from different receptors within the same species.

The term orphan receptor as used herein refers to a family of approximately 69 receptors which have been identified by amino acid sequence homologies. The orphan receptor has an amino acid sequence that is highly conserved in the DNA binding domain, and has consensus regions located in the ligand binding domain of the receptor. They are called orphan receptors because no ligand has been identified that directly activates any of the members of the family. An example of an orphan receptor is the chicken ovalbumin upstream promoter transcription factor (COUP-TF).

As used herein, the terms “metabolic bone disease”, “orthopedic bone disease” or “dental disease” are defined as conditions characterized by decreased bone mass and/or structural deterioration of the skeleton and/or teeth.

As used herein, the term “apoptosis” refers to programmed cell death characterized by nuclear fragmentation and cell shrinkage as detected by morphological criteria and Terminal Uridine Deoxynucleotidal Transferase Nick End Labeling (TUNEL) staining.

The term “host”, as used herein, refers to any steroid receptor containing cell or animal.

The term “transgene” is used herein to describe genetic material which has been or is about to be artificially inserted into the genome of a mammal, particularly a mammalian cell of a living animal.

“Transgenic animal” means a non-human animal, usually a mammal (e.g., mouse, rat, rabbit, hamster, etc.), having a non-endogenous (i.e., heterologous) nucleic acid sequence present as an extrachromosomal element in a portion of its cells or stably integrated into its germ line DNA (i.e., in the genotropic sequence of most or all of its cells). Heterologous nucleic acid is introduced into the germ line of such transgenic animals by genetic manipulation of, for example, embryos or embryonic stem cells of the host animal.

“Construct” means a recombinant nucleic acid, generally recombinant DNA, that has been generated for the purpose of the expression of a specific nucleotide sequence(s), or is to be used in the construction of other recombinant nucleotide sequences.

“Operably linked” means a DNA sequence and a regulatory sequence(s) are connected in such a way as to permit gene expression when the appropriate molecules (e.g., transcriptional activator proteins) are bound to the regulatory sequence(s).

“Operatively inserted” means a nucleotide sequence of interest is positioned adjacent a nucleotide sequence that directs transcription and translation of the introduced nucleotide sequence of interest (i.e., facilitates the production of, e.g., a polypeptide encoded by an APP sequence).

“Signal transduction pathway responsive transcriptional control unit” means a regulatory DNA sequence that permits gene expression when transcriptional activator proteins are bound to the regulatory sequences in response to the activation of a signal transduction pathway including but not limited to MAPK, nuclear factor of κB cells (NFκB), JNK, AP-1, p38K, PKA, PKC, P13-K, or NFAT signal transduction pathways. The transcriptional activator proteins of signal transduction pathway responsive transcriptional control units can be phosphorylated by a protein kinase in the signal transduction pathway such that the activator proteins can then bind to the DNA regulatory sequence. Exemplary Signal transduction pathway responsive transcriptional control unit include but are not limited to serum response element, Activator protein 1, cAMP response element, E-box DNA binding element, E2F DNA binding element, glucocorticoid response element, heat shock response element, interferon γ activation sequence, interferon stimulated response element, nuclear factor of activated T cells, nuclear factor of κB cells, p53 response element, Rb response element, and STAT3 response element.

The term “reporter gene” as used herein refers to any of the variety of genes which produce a protein when transcription is activated by a test compound or chemical signal. This protein is measured to determine the effect on transcription.

Reporter genes are operably linked to a promoter under the control of steroid response element such as the estrogen response element or the like.

“Nongenotropic activity of steroid receptors” means rapid activation only of the ligand-binding domain of the receptor without the need for activation of the DNA-binding domain of the recepptor or interaction with accessory transcriptional regulators.

“Genotropic activity of steroid receptors” means ligand dependent modulation of transcription which is sensitive to inhibitors of transcription and translation.

A “steroid receptor related disorder” is meant to mean any human or animal disorder, condition, or disease caused by or characterized by abnormally high or low concentrations, availability, mutations or other imbalance of a steroid or steroid receptor. By “abnormally high” is meant any difference above normal sufficient to be manifested by physiological, biochemical, physical, mental, or psychological effects. By “abnormally low” is meant any difference below normal sufficient to be manifested by physiological, biochemical, physical, mental, or psychological effects. In a preferred embodiment, the steroid receptor related disorder is one wherein additional steroid transcriptional activity is not desired such as with disorders in postmenopausal women. Steroid receptor related disorders can be associated with any cell, tissue, organ, or system that expresses steroid hormone receptors.

II. Method for Selectively Inducing a Steroidal Response

Based on this pioneering discovery, a method is provided of dissociating a steroidal nongenotropic effect from the steroidal genotropic effect of a steroid receptor. In one embodiment, the invention includes a method for selectively inducing a steroidal nongenotropic effect, that includes contacting the receptor with a compound that sufficiently interacts with the ligand binding domain of the receptor in a manner that causes the receptor to mediate a nongenotropic effect, while not activating the DNA-binding domain in a manner that induces a significant genotropic effect. In an alternative embodiment, a method is provided for selectively inducing a steroidal genotropic effect, that includes contacting the receptor with a compound that sufficiently activates the DNA-binding domain of the receptor in a manner that causes the receptor to mediate a genotropic effect, while not inducing a significant nongenotropic effect. It is considered that a ligand does not induce a “significant” or “substantial” effect if the effect is less than 10% that of the natural steroidal effect, and in a preferred embodiment, less than 5, 1, or 0.1 percent that of the natural steroidal effect.

Exemplary forms of nongentropic activity include but are not limited to intracellular second messenger systems, signal transduction pathways, preferably protein kinase signal transduction pathways, MAP kinase signal transduction pathways, Src/Shc/ERK signal transduction pathways; regulation of intracellular calcium concentration; secretion; changes in cellular morphology; cell motility; cytoskeletal rearrangements; and apoptosis.

In another aspect of the invention, a selectivity inducing compound (including but not limited to a peptide or protein or a ligand antagonist) can be administered that competes with an endogenous or exogenous ligand of a steroid receptor for a binding site on the receptor as a means to mask the activity of that ligand-binding interaction. As one example, a peptide or ligand antagonist can be administered that binds with the ligand binding domain in a way that prevents a ligand from inducing a nongenotropic effect. In an alternative embodiment, a peptide or ligand antagonist can be administered that inactivates the DNA-binding domain in a way that prevents the ligand from inducing a genotropic effect. The selectivity-inducing compound can inhibit the transcriptional activity of the steroid receptor directly by interfering with DNA-receptor interactions or indirectly by preventing the receptor from forming a protein-protein interaction with either the ligand binding domain or the DNA binding domain, as desired. Additionally, the selectivity inducing compound can exist as a complex having at least two components such that when the components combine, the complex inactivates the genotropic activity and activates the nongentropic activity of a steroid receptor.

In a still another aspect, a method for inducing a selective effect of a steroid receptor is provided that includes administering an effective amount of a compound that inactivates the DNA binding region of the receptor by inducing a conformational or configurational change in the receptor. In yet another aspect of the invention, a method for inducing a selective effect of a steroid receptor is provided that includes administering an effective amount of a compound that inactivates functions mediated only by the ligand binding region of the receptor by inducing a conformational or configurational change in the receptor. As used herein, the term confornational change refers to a change in the three dimensional structure of the receptor. The term configurational change refers to a chemical change in the protein structure.

This invention can be used to selectively effect a response in any of the family of steroid receptors, including those set out in Table 1 herein, and in particular, the α or β form of the estrogen receptors (ERα and ERβ), the estrogen receptor related receptor-1 and receptor-2 (ERR-1 and ERR-2), the androgen receptor (AR), the progesterone receptor (PR), retinoic acid receptor (and related orphan receptors RORα, RORβ, and ROR-γ); glucocorticoid receptor (GR), mineralcorticoid receptor (MR), Vitamin D receptor, neuroactive receptor, famesoid X receptor (FXR), liver X receptor (LXRα and LXRβ), thyroid hormone receptors A and B (which bind triiodothyronine (T3) and thyroxine (T4)), COUP-TF, ecdysone, the PPAR family (peroxisome proliferator activated protein, including PPARα, PPARγ, and PPARβ), pregnane X (“PXR”), bile acid binding family, and chimeric and orphan receptors thereof.

III. Induction of Nongenotropic Effect by Steroid through Non-related Receptor

In another embodiment of the invention, it has been discovered that steroids can induce a nongenotropic effect via an interaction with a steroid receptor other than its typical binding receptor. As used herein, the term “non related receptor” refers to one through which the steroid does not induce a genotropic effect. It was heretofor unknown that a steroid can act through an unrelated receptor, because it by definition does not induce a genotropic effect through an unrelated receptor, and the cellular nongenotropic effects were unclear at best. This invention thus includes a method to selectively induce a nongenotropic effect of a steroid receptor, comprising contacting the receptor with an unrelated steroid that sufficiently binds to and activates a function of the steroid ligand-binding domain to accomplish the desired effect. Any of the method of screening methods presented herein can be used to determine whether a selected steroid can induce a target nongenotropic effect via a previously considered unrelated steroid receptor.

In one nonlimiting illustration of this aspect of the invention, it has been discovered that the estrogen receptors (ERα and ERβ) and the androgen receptor (AR) can transmit anti-apoptotic signals to osteoblasts and osteocytes with similar efficiency irrespective of whether the binding ligand is an estrogen or an androgen. Further, it has been discovered that the nongentropic receptor-dependent mechanism of action of estrogens and androgens, which in the case of osteoblasts/osteocytes involves activation of extracellular signal regulated kinases (ERKs) are mechanistically dissociable from the transcriptional activity of the receptors.

The fact that a steroid can use a previously considered unrelated steroid receptor to induce a nongenotropic effect now explains the anomaly described in the Background of the Invention that nongenotropic steroid effects occur in cells or tissues devoid of the respective classic receptor. In such a case, the steroid is using an unrelated steroid receptor to mediate that nongenotropic effect (with the exception of course of the event in which the effect is not receptor mediated at all).

IV. Method for Screening a Compound for its Ability to Selectively Induce a Steroidal Response

A method is provided for screening compounds that are capable of inducing a steroidal nongenotropic effect without substantially inducing a steroidal genotropic effect or are capable of inducing a steroidal genotropic effect without substantially inducing a steroidal nongenotropic effect. The method can be carried out in a number of ways, including:

(1) assessing the ability of the compound to appropriately bind to the ligand-binding domain of the steroid receptor without substantially activating the DNA-binding domain of the steroid receptor, or to appropriately activate the DNA-binding domain of the steroid receptor without substantially activating functions mediated by the ligand-binding domain of the steroid receptor; and then

(2) measuring the biological activity of the test compound to assess its ability to induce a target nongenotropic effect without substantially inducing a genotropic effect, or its ability to induce a target genotropic effect without substantially inducing a genotropic effect.

The first step can be accomplished via molecular modeling, preferably with the aid of a computer. Alternatively, modified forms of the receptor lacking a functional DNA binding domain can be used to determine whether the test compound actvates only the ligand binding domain or only the DNA binding domain. The modified form can be expressed, for example, in a transformed cell. Cells cultured in vitro can be genetically altered to express a steroid receptor or a genetic variant thereof. Genotropic activity can be quantified using known techniques such as measuring the amount of total mRNA generated in response to a test compound or the mRNA specific for one or more steroid regulated genes. Screening can be done in vivo or in vitro

Alternatively, steps 1 and 2 can be combined by simply assaying for compounds that selectively induce either a nongenotropic or genotropic effect without substantially inducing the other effect in an in vitro cell-based assay.

An additional embodiment of the present invention is a method of making a tissue culture screening system comprising the steps of growing a tissue culture cell line until cell growth has reached appropriate confluence, typically approximately 50% confluence; transfecting the tissue culture with an appropriate expression plasmid of a specific receptor and an expression plasmid for a target gene responsive to the selected receptor; contacting the transfected cells with a test compound; and

then determining the effect of the test compound on the expression of the target gene to determine whether the compound induces a genotropic effect. In an aspect of this embodiment, it is also assessed whether the compound has induced a desired nongenotropic response. As a nonlimiting example of this embodiment, cells are contacted with a pro-apoptotic agent; the number of apoptotic transfected cells contacted with the test compound is determined; and the compounds that inhibit apoptosis and do not substantially induce the transcription of the target gene are selected.

In the screening methods described herein a tissue culture cell line can be devoid of the receptor whose biological activity is being tested. In this case the missing receptor is added to the cell line by transfection of a plasmid carrying the cDNA for the missing receptor or a chimera for the deficient receptor. The response element for that receptor can also be added via transfection.

The receptor responsive enhancer DNA element is selected from the responsive elements corresponding to the specific receptor used in the system.

The basal promoter consists of a minimal promoter sequence that contains a “TATA” element capable of binding RNA polymerase II and is selected from any of the commonly used elements. For example, thymidine kinase promoter, ovalbumin promoter and MMTV LTR.

The reporter gene can be selected from any of the variety of genes in which the protein product is easily assayed or detected. One skilled in the art readily recognizes that the protein can be easily assayed by calorimetric, fluorescent; immunochemical, chemical or radiochemical methods. Further, the reporter gene can be a chimera. Examples of various reporter genes which can be used include the bacterial enzyme chloramphenicol acetyl transferase (CAT), β-galactosidase, alkaline phosphatase, luciferase, peptide hormones, growth factors and chimeric proteins. In the preferred embodiment genes producing non-native proteins are used. For example, CAT is used in mammalian systems because it is not a normally occurring enzyme in mammalian cells and is easy to assay. Thus, the receptor dependent activation of CAT in tissue culture cells can be used to identify compounds which directly or indirectly interact with the receptor to activate transcription of a gene.

In another embodiment, a method for screening compounds for the treatment of steroid receptor related diseases or disorders is provided comprising: (i) contacting a cell expressing a natural or artificial steroid receptor with a test compound; (ii) assessing whether the compound activates a nongentropic activity of the steroid receptor, including but not limited to an intracellular second messenger system, signal transduction pathway, protein kinase signal transduction pathway, MAP kinase signal transduction pathway, Src/Shc/ERK signal transduction pathway; regulation of intracellular calcium concentration; secretion; changes in cellular morphology; cell motility; cytoskeletal rearrangements; or apoptosis; (iii) determining the level of transcription induced by the test compound; and (iv) selecting the compound or compounds that activate the nongentropic activity of the steroid receptor without substantially activating the genotropic activity of the steroid receptor. “Substantially activating” the genotropic activity of a steroid receptor means activating transcription more than 10% that induced by the natural steroid receptors, and preferably, less than 5, 2, 1 or 0.1%.

In another embodiment, a method for screening compounds for treatment of a sex steroid receptor related diseases or disorder is provided comprising: (i) contacting a cell expressing a natural or artificial sex steroid receptor with a test compound alone or in combination with a pro-apoptotic agent; (ii) assessing whether the compound activates a nongentropic activity of the steroid receptor, for example, a signal transduction pathway such as a protein kinase signal transduction pathway, a MAP kinase signal transduction pathway, or in general a mechanism that prevents apoptosis; (iii) determining the level of transcription induced by the test compound; (iv) determining whether the test compound inhibits apoptosis in cells contacted with a test compound alone or in combination with a pro-apoptotic agent; and (v) selecting the compound or compounds that activate the nongentropic activity of the steroid receptor without substantially activating the genotropic activity of the steroid receptor.

In yet another embodiment, a method for the selection of a compound useful in the treatment of bone disease or to increase bone mass is provided that includes assessing whether a compound activates the anti-apoptotic domain of the estrogen or androgen receptor without substantially activating the DNA binding domain of the receptor, using methods as described further herein or otherwise known. In a preferred embodiment, the steroid hormone receptor protein is expressed from endogenous DNA. In another embodiment the receptor sequence can be human, non-human, or chimeric. By chimeric it is meant, a resulting sequence comprised of a mixture of sequences from at least two different sources. Alternatively, the receptor can be genetically altered such that specific sequences are deleted, added, or modified.

In another embodiment, the activation of the DNA binding domain of a steroid hormone receptor is determined by using a non-endogenous DNA sequence encoding an operable hormone response element operably linked to a reporter gene. Methods of measuring transcription levels are known in the art and include physical isolation and quantification of mRNA as well as more complex procedures. For example, the production of mRNA can be quantified using reverse transcription-polymerase chain reaction (RT-PCR). When optimally performed, RT-PCR can be used to detect transcripts produced at very low levels and can identify RNAs in minute quantities of starting material. Reagents and RT-PCR kits are commercially available. Thus, endogenous mRNA levels can be readily monitored to assess the effect of a compound on transcription levels. In other embodiments of the present invention, compounds are not screened by assaying changes in the appearance or physical characteristics of a compound in response to a test compound interacting with the steroid receptor.

Alternatively, the expression of genes can be determined using DNA microarray assays. Briefly, nucleic acid, in the form of long double stranded complimentary DNAs or oligonucleotides, is applied to glass microscope slides, either using robot controlled pins or solid phase chemical synthesis. Tens to hundreds of thousands of unique spots of nucleic acid can be applied to each slide. Radioactively or fluorescently labeled sample (probe) DNA is then applied to the slide and after an appropriate period of time, and several washing steps, the hybridized probe nucleic acid is detected using a microscope or scanner. If the nucleic acid identity of each spot on the slide is known, the corresponding nucleic acid in the sample can be detected by the presence of a signal at that spot's location on the slide. Using this method the expression level of thousands of genes can be simultaneously measured in just a few hours.

In still another embodiment, activators of selective nongentropic signalling can be selected by (i) contacting cells expressing a natural or artificial steroid receptor(s) with a test compound; (ii) determining the amount of ERK activation in the cells contacted with the test compound; (iii) determining the amount of transcription in the cells contacted with the test compound; (iv) selecting the cells contacted with the test compound that exhibit ERK activation and minimal transcription levels as compared to transcriptions levels in cells contacted with the natural steroid. In a preferred embodiment, the compounds are identified using a high throughput combination screening which simultaneously detects ERK activation using a SRE-SEAP construct (positive readout) and C3 (complement 3) transcription (negative readout).

The SRE-SEAP assay detects activation of the MAPK/JNK signalling pathways by means of assaying transcription factor (Elk-1/SRF)—induced activation of a Serum Response Element (SRE) placed upstream of the herpes simplex virus thymidine kinase (HSV-tk) promoter that drives the expression of secreted alkaline phosphatase (SEAP). This assay is commercially available as are kits for in vivo assays for MEK1 or MEKK1. In general, 24 hrs after transfection serum-containing medium is replaced with medium that contains about 0.2% serum. Cells are cultured for 24 hrs in about 0.2% serum and then assayed. Control samples are treated with vehicle alone.

A further embodiment of the present invention is an assay for identifying a test compound or chemical signal that activates nongentropic receptor activity without substantially activating genotropic receptor activity, comprising the steps of growing a tissue culture screening system in appropriate media, wherein the cell line of the tissue culture screening system contains a steroid receptor having at least one identified nongentropic activity; adding a test compound or chemical signal to the media and measuring the amount of transcription and the amount of nongentropic activity. One skilled in the art will readily recognize that the media chosen depends on the cell line chosen and can be any of the chemically defined culture media known in the art and suitable for the cell line. The receptors and reporter genes can be added by either transient or stable transfection.

In another embodiment, a method for screening for a selective ligand of a steroid receptors is provided comprising the steps of: (i) culturing cells in cell culture media wherein the cells naturally or artificially express a steroid receptor; (ii) contacting the cells with a test compound; (iii) determining whether the test compound activates nongentropic activity of the steroid receptor; (iv) determining whether the test compound activates genotropic activity of the steroid receptor; (v) selecting the compound that activates nongentropic activity but does not substantially activate genotropic activity of the steroid receptor.

In one embodiment, the cell line is OB-6 and the nongentropic activity can be determined by assessing whether the test compound inhibits apoptosis. Apoptosis can be assessed using techniques known in the art. For example, cells can be transfected with Green Fluorescent Protein localized to the nucleus to assist in assessing changes in nuclear morphology correlating to apoptosis. Apoptosis can also be determined using Terminal Uridine Deoxynucleotidal Transferase Nick End Labeling, In Situ End Labeling, Hoechst staining, or assessing caspase-3 activity. Additionally, nongentropic activity can be assessed by determining whether a signal transduction pathway has been activated, preferably the Src/Shc/ERK signal transduction pathway. Cells can be transfected with a nongentropic activity marker, preferably the serum response element/secreted alkaline phosphatase (SRE-SEAP) construct such that the induced activation of the serum response element drives the expression of secreted alkaline phosphatase. The SRE-SEAP construct detects the activation of the MAPKIJNK signalling pathways by means of assaying transcription factor (Elk-1/SRF). The degree of genotropic activity can be assessed using techniques known in the art. In preferred embodiments, steroid receptors contacted with a test compound can be isolated and combined with a nucleic acid sequences corresponding to the steroid response element associated with the particular steroid receptor. Lack of binding of the steroid-test compound complex to the response element sequence indicates that the test compound does not affect genotropic activity. One of ordinary skill in the art will appreciated that the either the unlabeled steroid or the unlabeled response element can be immobilized on a solid phase and a labeled steroid or labeled response element as needed can then be added to the solid phase. Detection of the label on the solid phase in the presence of the test compound indicates that the test compound-receptor complex does not bind nucleic acid and therefore, does not affect genotropic activity. Alternatively, the steroid receptor-test compound complex can be used in binding assays with known nuclear transcription factors including but not limited to AP-1, NFkB, SRC-1 and the like. Lack of binding of the steroid receptor-test compound with transcription factors indicates that the test compound does not inhibit genotropic activity.

Additional embodiments of the present invention include screening methods using multiple transfectants such that cells can be optionally transfected with steroid receptors, inducible reporter gene plasmids for detecting genotropic activity; or inducible reporter gene plasmids for detecting nongentropic activity. The steroid receptor is transcriptionally active such that it can induce the expression of one or more reporter genes. Compounds can be screened for desired effects on genotropic or nongentropic activity by monitoring the change in reporter gene expression when transfected cells are treated with a test compound. For example, a first reporter gene construct can have a response element upstream of a reporter gene such that the transfected steroid receptor encodes a protein that interacts with the steroid response element to induce the expression of the reporter gene. The cells can be single, double, or triple transfectants, or any other multiple of transfectants. In another embodiment, a method of screening for ligands of steroid receptors that induce nongentropic activity without substantially inducing genotropic activity is provided comprising the steps of: (a) contacting a cell with a test compound wherein the cell has been transfected with: i) a DNA sequence encoding a functional steroid receptor or genetic variant of a steroid receptor wherein the steroid receptor is transcriptionally active; ii) an response element-reporter gene construct; and iii) serum response element-reporter gene construct; (b) determining the effect of the test compound on the transcription of the response element-reporter gene construct; (c) determining the effect of the test compound on the transcription of the serum response element-reporter gene construct; and (d) selecting the compound that activates the transcription of the serum response element-reporter gene construct without substantially effecting the transcription of the ERE-reporter gene construct.

Those of ordinary skill in the art will recognize that the invention can be practiced using multiple cell lines rather than a single cell line containing multiple transfectants. In one embodiment, a method of screening for compounds to treat steroid receptor related disorders is provided comprising; providing a first cell line transfected with a transcriptionally active steroid receptor and first reporter-gene construct transcriptionally responsive to the steroid receptor; providing a second cell line transfected with the transcriptionally active steroid receptor and a second reporter gene construct wherein transcription of the second reporter gene construct indicates the activation of nongentropic activity, preferably a signal transduction pathway; determining the effect of a test compound on the transcription of the first and second reporter gene constructs; and selecting the compound that induces the activation of the second reporter gene construct without substantially activating the transcription of the first reporter gene construct.

In yet another embodiment of the present invention, a method of screening for compounds effective for the treatment of steroid receptor related diseases or disorders is provided comprising; administering a test compound to a host, preferably a mammal, most preferably a mouse; determining the effect of the compound on the target nongenotropic action; determining the effect of the compound on transcription of genes regulated by the steroid receptor, and selecting the compound that mediates the nongenotropic effect and does not substantially activate the transcription of steroid regulated genes.

Apoptosis of bone cells can be assessed by obtaining bone cells from the host after administration of the test compound using TUNEL or Hoechst staining. Additionally, bone histomorphometry can be used for directly and precisely analyzing the effect of test compounds on bone tissue. Histology samples are usually obtained by bone biopsy. The effects of a test compound on the apoptosis of bone cells can also be assessed by determining the effect of the test compound on bone mass or bone strength. Bone mass can be assessed using DEXA or peripheral quantitative computed tomography (pQCT). Bone strength can be measured with biomechanical testing. The activation of transcription of steroid regulated genes can be assessed by determining the effect of a test compound on uterotrophic activity or breast activity.

Those of ordinary skill in the art will appreciate that the screening methods of the present invention can also be utilized to identify compounds that activate genotropic steroid receptor activity without activating nongentropic steroid activity. These compounds can be used when nongentropic activity of steroid receptors is unwanted but genotropic activity is desired. Thus, these compounds can be used to treat steroid or steroid receptor related diseases and disorders where genotropic activity of the steroid is desired but the nongentropic activity is not. Thus, in one embodiment, a method for screening compounds for the treatment of steroid receptor related diseases or disorders, in one embodiment a sex steroid receptor related diseases, is provided comprising: (i) contacting a cell expressing a natural or artificial steroid receptor, in one embodiment a sex steroid receptor, with a test compound; (ii) assessing whether the compound activates a nongentropic activity of the steroid receptor; (iii) determining the level of transcription induced by the test compound; and (iv) selecting the compound or compounds that activate the genotropic activity of the steroid receptor without substantially activating the nongentropic activity of the steroid receptor. Substantially activating the nongentropic activity of a steroid receptor means activating a second messenger system such that the second messenger system induces a nongenotropic biological response in the cell. Exemplary biological responses are chemical cascades, including but not limited to those caused by phosphorylation, changes in morphology, secretion, proliferation, DNA synthesis, protein synthesis, and cytoskeletal rearrangements.

V. Methods for Using Compounds That Induce Selective Steroid Receptor Activity

The active compounds which satisfy the criteria set out in detain herein can be used to treat a wide variety of medical conditions. Moreover, because steroid receptors including sex steroid receptors are known to be expressed on a variety of tissues in mammals including but not limited to nervous tissue, muscle tissue, bone tissue, blood, reproductive tissues, the active compounds of the present invention can be used to treat a target disorder of these or other tissues wherein the disorder is related to steroid action, either genotropic or nongentropic.

In a selected embodiment, the active compounds can be used to treat cardiovascular disease. For example, compounds having the nongentropic steroid activity of relaxing smooth muscle in large and small arteries can be used to modulate vasoreactivity and sympathetic balance. Active compounds identified by the present invention may be active on multiple steroid receptors and may have different effects on each. For example, an active compound could inhibit the genotropic activity of one steroid receptor without inhibiting the genotropic activity of a different steroid receptor. Similarly, the nongentropic activity of steroid receptor may be inhibit while the nongentropic activity of a different steroid hormone receptor is activated.

The active compounds of the present invention may also be used to treat endocrine disorders including non-insulin-dependent diabetes as well as to modulate the immune system. T cells are known to express steroid receptors and the active compounds may be used to treat proliferative and infectious diseases. The active compounds can also be used to treat neurological disorders including epilepsy, anxiety, depression, insomnia, migraine, memory impairment, and drug dependency.

The active compounds can be used as bone anabolic agents in a host, including a human, to strengthen bone for strenuous physical activities such as sports or manual labor, and to strengthen bone in persons or other hosts who do not have osteoporosis but might be subject to osteoporosis in the future because the host is in a risk group for that disease. Other uses for a bone anabolic agent in humans include the treatment of hosts, including persons who are born with naturally thin, small, or unusually fragile bones, including weak teeth, persons who have a genetic predisposition to a bone catabolic disease, or an orthopedic bone disease such as joint degeneration, non-union fractures, orthopedic problems caused by diabetes, periimplantitis, poor responses to bone grafts, implants, or fracture.

These compounds can be used to increase the bone mass in horses and dogs used for labor as well as those used in sports such as racing. The compounds can also be used to increase the bone mass in chickens and turkeys used in meat production to increase the ease of processing.

Representative metabolic bone diseases are postmenopausal osteoporosis, senile osteoporosis in males and females, glucocorticoid-induced osteoporosis, immobilization-induced osteoporosis, weightlessness-induced osteoporosis (as in space flights), post-transplantation osteoporosis, migratory osteoporosis, idiopathic osteoporosis, juvenile osteoporosis, Paget's Disease, osteogenesis imperfecta, chronic hyperparathyroidism, hyperthyroidism, rheumatoid arthritis, Gorham-Stout disease, McCune-Albright syndrome and osteolytic metastases of various cancers or multiple myeloma. Characteristics of the orthopedic bone diseases are loss of bone mass, general bone fragility, joint degeneration, non-union fractures, orthopedic and dental problems caused by diabetes, periimplantitis, poor responses to bone grafts/implants/bone substitute materials, periodontal diseases, and skeletal aging and its consequences.

Compounds selected according to the criteria provided herein can also be used for the augmentation of bone mass and/or fracture prevention in diseases characterized by low bone mass and increased fragility. The compounds can also be used to treat bone disease states in which osteoblastogenesis is decreased, such as senile osteoporosis, and glucocorticoid-induced osteoporosis—especially in growing children and adolescents, during which time in whom interfering with bone remodeling is detrimental. Moreover, those of ordinary skill in the art will recognize that the compounds identified by the present invention can be utilized to treat disease states in which steroid therapy in general, preferably sex steroid therapy, is indicated. Conditions where steroid therapy is indicate include inflammation, arthritis, lupus erythematosus, organ transplants, cancer, epilepsy, anxiety, depression, insomnia, migraine, memory impairment, and drug dependency, immune disorders, and cardiovascular disoreders.

The compounds selected by the present invention are advantageous over existing steroid therapies because the selected compounds can initiate the signal transduction mechanism of the steroid hormone receptor pathway and the benefits thereof without inducing transcription thereby preventing adverse side effects associated with current steroid therapy. Examples of side effects associated with current sex steroid therapy include unwanted secondary sexual characteristics including hirsutism in females and gynecomastia in males etc. Compounds exhibiting varying levels of transcriptional activity and anti-apoptotic activity can be useful for the treatment of different conditions or diseases, and those of ordinary skill in the art can readily determine the level of transcription and anti-apoptotic activity of a compound needed to treat a specific disease using methods commonly known in the art and disclosed herein. In other embodiments, active compounds can be selected that activate the genotropic activity of a steroid receptor but do not activate the nongentropic activity of the steroid receptor. These active compounds are advantageous when nongentropic activity of steroid receptors is undesired.

VI. Kits

An additional embodiment of the present invention is a kit for measuring steroid receptor dependent nongentropic activity comprising: a container having a stable transfected cell line wherein the cell line includes a steroid responsive reporter gene construct, a means for assessing the activation of nongentropic steroid activity, and a steroid receptor. The kit can also include a cell line in which at least one of the steroid receptors, the reporter gene construct is included as an expression plasmid.

In one nonlimiting example, 17β-estradiol is a positive compound control of ERK activation as well as C3 transcription. Pyrazole can be used as negative control of ERK activation and the estren as a negative control of C3 transcription. Compounds with strong SRE-SEAP activity but weak or absent C3-Luc activity, compared to 17β-Estradiol are selected. The selection criteria can include classifying compounds of interest based on the ratio of ERK activation over C3 transcription.

VII. Transgenic Animals

In still another embodiment of the present invention, a transgenic non-human animal is provided that includes an inducible transgene encoding at least one steroid receptor antagonist, for example, an ER peptide antagonist such as peptide F6, αII, or 293, and preferably two copies of the peptide are encoded. In one embodiment, induction of the transgene blocks both the genotropic and the nongenotropic of the receptor. In another embodiment, induction of one or two transgenes block the transcriptional activity of a steroid receptor. A transgenic non-human animal, preferably a mouse, having at least one inducible steroid receptor specific antagonist can be used to screen for compounds to treat disease that act specifically through that receptor.

In one embodiment, a method for screening for compounds to selectively induce a receptor response is provided comprising, (i) inducing the expression of at least one receptor antagonist in a transgenic animal containing at least one inducible receptor antagonist transgene; (ii) administering a test compound to the transgenic animal; (iii) determining the extent of nongenotropic effect in the animal of step (ii); (iv) determining the level of transcription in the animal of step (ii); and (v) selecting the compound that induces the nongenotropic effect without significantly increasing transcription in the transgenic animal administered the test compound. The creation of transgenic animals is known in the art. U.S. Pat. No. 6,025,539 to Lee et al. discloses an IL-5 transgenic mouse and method for producing transgenic mice and is incorporated herein in its entirety. One of ordinary skill in the art will appreciate that inducible transgenes can be cell, tissue, or organ specific. In one embodiment, the transgene is under the control of the reverse tetracycline repressor/tetracycline -ON system driven by the actin promoter and as described in Gossen, M. et al. (1993) Trends Biochem Sci 18: 471-475; and Gossen M. et al. (1995) Science 268:1766-1769.

In a more specific embodiment, a method for screening for compounds to selectively induce a receptor response is provided comprising, (i) inducing the expression of at least one ER antagonist in a transgenic animal containing at least one inducible ER antagonist transgene; (ii) administering a test compound to the transgenic animal; (iii) determining the number of osteoblasts or osteocytes undergoing apoptosis in the animal of step (ii); (iv) determining the level of transcription in the animal of step (ii); and (v) selecting the compound that inhibits apoptosis without significantly increasing transcription in the transgenic animal administered the test compound.

The reverse tetracycline repressor (rTetR or TET-ON) system takes advantage of the high specificity of the tetracycline repressor (TetR)-operator-tetracycline (Tc) interaction, the herpes simplex virus (HSV)-VP16 transcription activation domain, and tetracycline (Tc). In the conventional (TET-OFF) system, the Tc-controlled transactivator (tTA), a fusion between TetR and the activating domain of the VP16 protein, stimulates gene expression, as has been shown with reporter assays in HeLa cells. Tc can prevent tTA from binding to tet operators (tetO) upstream of a minimal promoter, and consequently turns off tTA-dependent expression. However, the TET-ON system utilizes a rtTA transactivator that has been modified, such as Tc, or its derivative Doxycycline (Dox) that induces rather than abolishes binding of VP16 to the operator. The TET-ON system circumvents any potential toxic effects that may be conferred to eukaryotic cells following prolonged exposure to tetracycline.

In another embodiment, a transgenic non-human animal is provided that has an inducibly inactivated steroid receptor. In one embodiment, a transgenic animal is disclosed in which the sex steroid receptor is inactivated only in osteoblasts and osteocytes. The Cre/loxP binary recombination system is a preferred method for generating these transgenic animals. Identified transgenic animals can be characterized in terms of their ability to switch on Cre expression in response to Dox. In another embodiment, a transgenic animal is provided wherein specific exons of the steroid receptor gene are inducibly spliced creating specific steroid receptor deletions. In one example, exon 3 or exon 4 of the ERα gene is inducibly spliced to generate a transgenic animal having an ERα deletion in exon 3 or exon 4 or both exons.

In still another aspect of the present invention, a non-human transgenic animal model of bone disease is provided. The transgenic animal can be heterozygous or homozygous for the transgene and can have one or more transgenes. The transgene can be operably linked to an inducible promoter. In other aspects of the invention, a transgenic animal is provided that includes a sex steroid receptor that (i) contains a DNA binding domain that does not mediate a genotropic sex effect on exposure to an estrogen or androgen; (ii) contains a ligand binding domain that does not induce an anti-apoptotic effect on osteoblasts or osteocytes on exposure to an estrogen or androgen; or (iii) or contains both (i) and (ii).

In yet another aspect, at least one polynucleotide used as the transgene to block both the genotropic and the anti-apoptotic activity of the estrogen receptor but not the androgen receptor. For example, using 2XF6 as the transgene, the activity of the estrogen receptor can be blocked but the anti-apoptotic activity of estrogen or androgen through the androgen receptor can be retained.

VIII. Combination Therapy

In one aspect of the invention, one of the active compounds described herein can be administered to a host to treat steroid or steroid receptor related diseases or disorders including bone disease in combination with a second pharmaceutical agent.

With respect to bone disease, the second pharmaceutical agent can be a bone anti-resorption agent, a second bone mass anabolizing agent, an antioxidant, a dietary supplement, or any other agent that increases the beneficial effect of the active compound on bone structure, strength, density, or mass. Any member of the ten classes of drugs that are used in the treatment of osteoporosis can be administered in combination with the primary active agent, including: an anabolic steroid, a bisphosphonate, a calcitonin, an estrogen or progesterone, an anti-estrogens such as raloxifene or tamoxifene, parathyroid hormone (“PTH”), fluoride, Vitamin D or a derivative thereof, or a calcium preparations.

Nonlimiting examples of suitable agents for combination include, but are not limited to, alendronic acid, disodium clondronate, disodium etidronate, disodium medronate, disodium oxidronate, disodium pamidronate, neridronic acid, risedronic acid, teriparatide acetate, tiludronic acid, ipriflavone, potassium bicarbonate, progestogen, a thiazide, gallium nitrate, NSAIDS, plicamycin, aluminum hydroxide, calcium acetate, calcium carbonate, calcium, magnesium carbonate, and sucralfate. Reducing agents, such as glutathione or other antioxidants may also be useful in combination with any of the compounds of the present invention. As used herein, the term antioxidant refers to a substance that prevents the oxidation of an oxidizable compound under physiological conditions. In one embodiment, a compound is considered an antioxidant for purposes of this disclosure if it reduces endogenous oxygen radicals in vitro. The antioxidant can be added to a cell extract under oxygenated conditions and the effect on an oxidizable compound evaluated. As nonlimiting examples, antioxidants scavenge oxygen, superoxide anions, hydrogen peroxide, superoxide radicals, lipooxide radicals, hydroxyl radicals, or bind to reactive metals to prevent oxidation damage to lipids, proteins, nucleic acids, etc.

In another aspect of the invention, one of the active compounds described herein can be administered to a host to treat a steroid receptor related disorder in combination with a second pharmaceutical agent. The second pharmaceutical agent can be a steroid, sex steroid, modified steroid, modified sex steroid, antibiotic, antioxidant, dietary supplement, or any other agent that increases the beneficial effect of the active compound on steroid receptor related disorders.

IX. Pharmaceutical Compositions

A compound that induces a selective steroidal effect or its pharmaceutically acceptable salt, selected according to the criteria described in detail herein, can be administered in an effective amount to treat any of the conditions described herein, optionally in a pharmaceutically acceptable carrier or diluent.

The active materials can be administered by any appropriate route for systemic, local or topical delivery, for example, orally, parenterally, intravenously, intradermally, subcutaneously, buccal, intranasal, inhalation, vaginal, rectal or topically, in liquid or solid form. Methods of administering the compound of the invention may be by specific dose or by controlled release vehicles.

A preferred mode of administration of the active compound is oral. Oral compositions will generally include an inert diluent or an edible carrier. The active compound can be enclosed in gelatin capsules or compressed into tablets. For the purpose of oral therapeutic administration, the compound can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; and/or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable derivative or salts thereof can also be mixed with other active materials that do not impair the desired action, or with materials that supplement the desired action, such as classical estrogen like 17β-estradiol or ethinyl estradiol; bisphosphonates like alendronate, etidronate, pamidronate, risedronate, tiludronate, zoledronate, cimadronate, clodronate, ibandronate, olpadronate, neridronate, EB-1053; calcitonin of salmon, eel or human origin; and anti-oxidants like glutathione, ascorbic acid or sodium bisulfite. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; chelating agents such as ethylenediaminetetraacetic acid (EDTA); buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In a preferred embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions (including liposomes targeted with monoclonal antibodies to surface antigens of specific cells) are also pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and/or cholesterol) in an inorganic solvent that is then evaporated, leaving behind a thin film of dried lipid on the surface of the container. An aqueous solution of the active compound or its monophosphate, diphosphate, and/or triphosphate derivative(s) is then introduced into the container. The container is then swirled by hand to free lipid material from the sides of the container and to disperse lipid aggregates, thereby forming the liposomal suspension.

The dose and dosage regimen will depend upon the nature of the metabolic bone disease, the characteristics of the particular active compound, e.g., its therapeutic index, the patient, the patient's history and other factors. The amount of an activator of nongentropic estrogen-like signaling compound administered will typically be in the range of about 1 pg/kg to about 10 mg/kg of patient weight. The schedule will be continued to optimize effectiveness while balanced against negative effects of treatment. See Remington's Pharmaceutical Science, 17th Ed. (1990) Mark Publishing Co., Easton, Pa.; and Goodman and Gilman's: The Pharmacological Basis of Therapeutics 8th Ed (1990) Pergamon Press.

For parenteral administration, the active compound will most typically be formulated in a unit dosage injectable form (solution, suspension, emulsion) in association with a pharmaceutically acceptable parenteral vehicle. Such vehicles are preferably non-toxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. An activator of nongentropic estrogen-like signaling compound will typically be formulated in such vehicles at concentrations of about 10 pg/ml to about 10 mg/ml.

The concentration of the compound in the drug composition will depend on absorption, inactivation, and excretion rates of the drug as well as other factors known to those of skill in the art. It is to be noted that dosage values will also vary with the severity of the condition to be alleviated. Additionally, the active ingredient may be administered at once, or may be divided into a number of smaller doses to be administered at varying intervals of time. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed composition.

X. Illustrative Examples

The following examples are given for the purpose of illustrating various embodiments of the invention and are not meant to limit the present invention in any fashion.

A. Estrogens and androgens modulate osteoclastogenesis via transcriptional regulation of cytokines; and osteoblastogenesis via attenuation of mesenchymal progenitor self-renewal. Estrogens and androgens also exert effects on the lifespan of mature bone cells: pro-apoptotic effects on osteoclasts, but anti-apoptotic effects on osteoblasts and osteocytes. The latter are mediated by a “nongentropic”, yet receptor-dependent, mechanism of action, and are mechanistically dissociable from transcriptional activity.

The potent anti-apoptotic effect of sex steroids on osteoblasts and osteocytes is mediated by a receptor-dependant mechanism and requires phosphorylation of ERKs via activation of Src and Shc kinases. Moreover, results in HeLa cells show that ERα, ERβ, or AR can transmit anti-apoptotic signals with the same efficiency irrespective of whether the ligand is an estrogen or an androgen. Using the ERα, as a paradigm, it has been discovered that the AF- 1 function, DNA binding, the F as well as the dimerization domains of this protein are dispensable for the anti-apoptotic effects of estrogen. Instead, these effects require the portion of the protein that resides within the E domain. Therefore, the anti-apoptotic activity of the estrogen receptor must be localized in a region of the protein that is distinct and dissociable from that which is responsible for the genotropic effects of the ligand-activated receptor. Evidence that the ability of the ER to convey the anti-apoptotic signal is lost by targeting the protein to the nucleus is also disclosed.

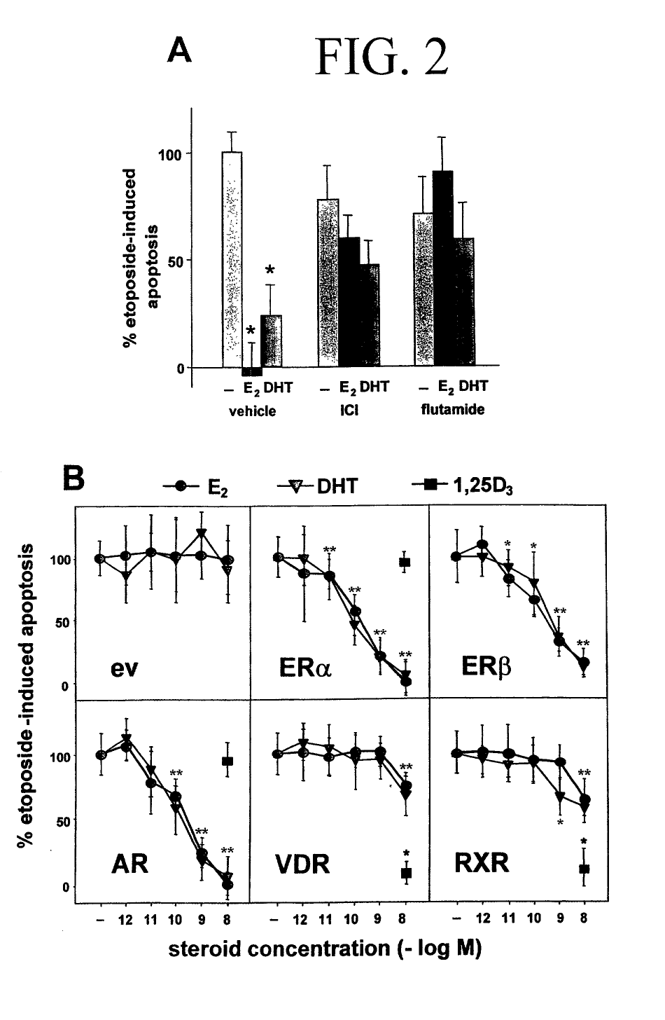

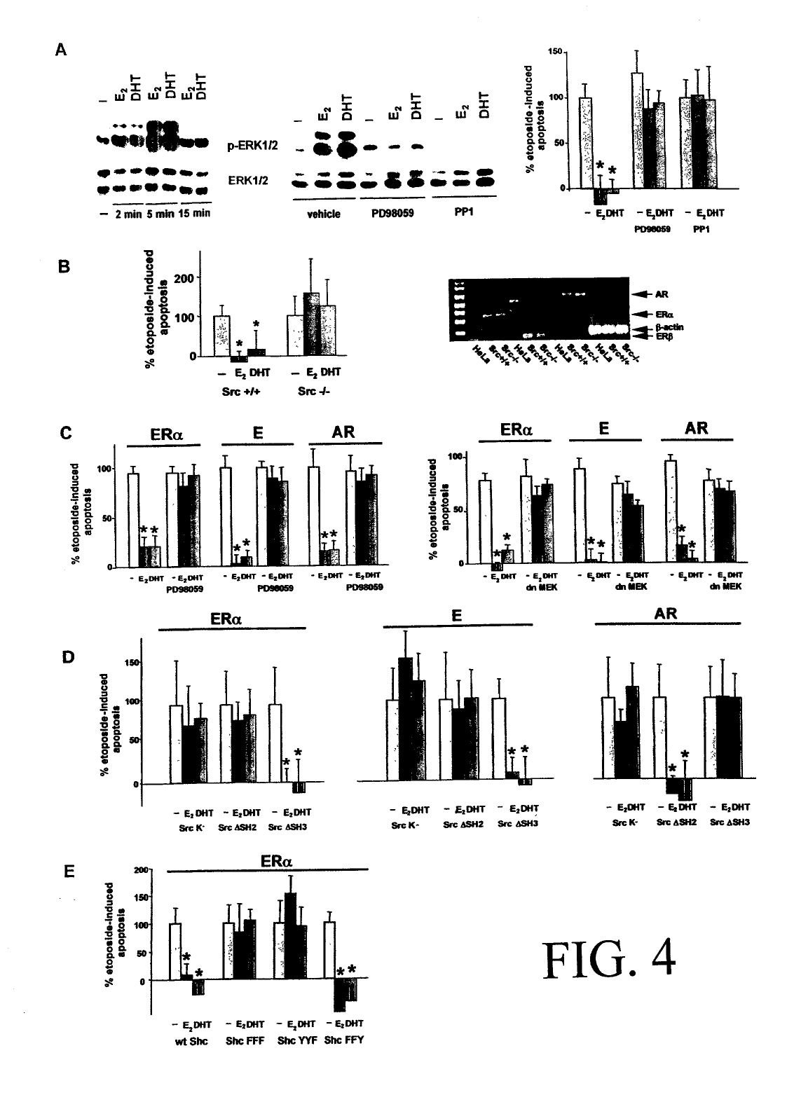

Others have reported biologic effects of the nuclear orphan receptor TR3 and ER which were independent of their transcriptional activity in the nucleus (Li, H. et al. (2000). Science 289, 1159-1164; Simoncini, T., et al (2000). Nature 407, 538-541). In addition, Migliiaccio et al. (Migliaccio, A., et al (2000). EMBO J. 19, 5406-5417) showed ER or AR co-immunprecipitate with Src in prostate cancer cells. Consistent with the finding that the anti-apoptotic activity of the ER requires the SH2 domain of Src, while the AR requires the SH3 domain, Migliaccio et al. also showed that ER interacted with SH2 domain and AR with the SH3 domain of Src. The present results (FIGS. 2 and 4) argue strongly that the cross-talk between antagonists, and more important the cross-talk between the cognate ligands-an issue that was not addressed in the Migliaccio study—is not dependant on the complexing of ER with AR, as prevention of apoptosis by the heterologous ligand could be demonstrated in cells transfected with only one steroid receptor.

Several members of the MAP kinase signaling pathway, including Src and ERKs, are clustered in caveolae—specialized membrane invaginations that are enriched in the scaffolding protein caveolin-1 and compartmentalize signal transduction (Okamoto,T., et al. (1998). J. Biol. Chem. 273, 5419-5422). Caveolae are found in a variety of cell types including human and murine osteoblasts (Solomon, K. R., et al. (2000) J. Bone Miner. Res. 15, 2391-2401; Solomon K. R., et al. (2000 J. Bone Miner. Res. 15, 2380-2390). ERα has been shown recently to co-immunprecipitate with caveolin-l (Schlegel, A., et al. (1999) J. Biol. Chem. 274, 33551-33556). A sub-population of ERα has been co-localized in caveolae with both caveolin and eNOS—an enzyme activated by estrogen through a nongentropic mechanism of action (Chambliss, K. L., et al. (2000) Circ. Res. 87, E44-E52). The dependency of the anti-apoptotic effects of estrogen and androgen on sex steroid receptors, along with the evidence that the anti-apoptotic signal of the E domain of the ER is preserved when targeting this protein to the membrane, but it is lost when targeting it to the nucleus, demonstrate that the effect can be mediated via a fraction of the classical receptors that is associated with the plasma membrane, probably within caveolae. This connection is supported by the finding of a co-dependency of the phenomena on Src, Shc and ERKs.

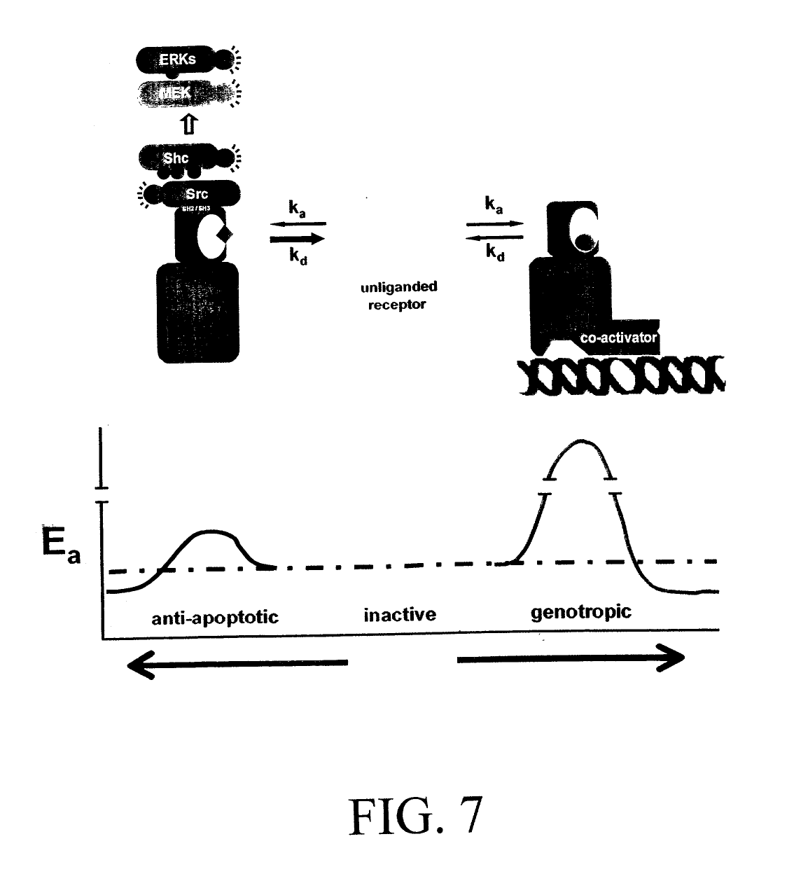

The data disclosed herein is the first demonstration that the nongentropic activity of a steroid receptor can be functionally dissociated from the genotropic activity of this transcription factor; and that this can be accomplished using synthetic steroidal or non-steroidal compounds. Although the invention has been exemplified using sex steroids, those of ordinary skill in the art will recognize that invention is applicable to all steroid hormones including estrogens, progestins, androgens, glucocorticoids and mineralcorticoids to separate nongentropic activity from genotropic activity. Similarly, the specification is the first to disclose novel treatments for bone disease using compounds that act through sex steroid receptors to inhibit apoptosis without inducing transcription. In one embodiment, ligand-induced dissociation of the two activities is the result of distinct conformational states, assumed by the receptor proteins following their physical association with the ligand, that are required to effect the genotropic versus the anti-apoptotic responses (FIG. 7). Because sex steroid receptors are found on many tissues other than reproductive tissues such as bone and brain, the compounds selected with the present invention can be used to treat conditions associated with the tissues or organs expressing sex steroid receptors. Exemplary tissues where sex steroid receptors have been found include but are not limited to pituitary and hypothalamic tissues, muscle tissue, blood cells, blood vessels, and colonic mucosa.

On the scale of molecular interactions and cellular events, a genotropic response is a relatively long-term one and thus is believed to require the classical ligand-receptor interaction involving a detailed fit to the hormone in the binding pocket to produce a complex of sufficient stability and lifetime to effect the multiple protein-protein and protein-nucleic acid interactions and the enzymatic activations that are required to remodel chromatin architecture and effect changes in the rate of gene transcription (FIG. 7, far right complex). The need to form such a stable, long-lived ligand-receptor complex places strict demands on ligand structure, leading to the well-known specificity and distinct pharmacology that characterize the classical genotropic effects of estrogens and androgens.

By contrast, ERK activation, which underlies the nongentropic anti-apoptotic response, is very rapid and may be effected by a less fully developed or broader range of conformations adopted by much more transient ligand-receptor complexes (FIG. 7, far left complex). The formation of such transient species might require merely that the ligand associates with the receptor briefly, and thus would be dominated by ligand association rates, rather than the ratio of association to dissociation rates that determines the affinity of ligands assessed by classical binding affinity assays. Because ligand association rates are much more comparable than dissociation rates (Bindal R. D., et al. (1987) J Steroid Biochem. 28, 361-370; Raynaud J P, et al. (1978) Cancer Res. 38, 3044-3050), the formation of such short-term complexes might show greatly relaxed structural specificity and potency/affinity ranking, as is manifest in the anti-apoptotic response.

The idea that a ligand-induced rapid response may exhibit a different potency order than a long-term response has interesting precedents. In the time course of events that follows estrogen stimulation of the uterus of immature rats, short-acting estrogens such as estradiol and dimethylstilbestrol and equipotent with long-acting estrogens such as estradiol and diethylstilbestrol in inducing early responses such as water imbibition and early RNA synthesis, whereas they are far less potent than the long-acting estrogens in inducing the later responses such as DNA synthesis and uterine growth (Katzenellenbogen B S, et al. (1979) Prog. Horm. Res: 35, 259-300; Katzenellenbogen B S, et al. (1978). Mol. Cell Endocrinol. 10, 103-113). In fact the term “impeded estrogen was coined to describe this characteristic change in potency between short-term and long-term responses. These differences were attributed to the need for continuous and long-term receptor occupancy by ligand to induce long-term responses, whereas transient occupancy was sufficient to induce the short-term ones.

Biologically more distant but more pertinent to the time scale of events described in this work is the concept of enzyme “slow, tight-binding inhibitors” (Szedlacsek, S. E. (1995) Methods Enzymol. 249, 144-180.; Morrison, J. F. (1988) Adv. Enzymol. Relat. Areas. Mol. Biol. 61, 201-301). In a number of cases, enzymes can be inhibited rapidly and effectively by a rather wide range of ligands, only a few of which then proceed slowly to form much more stable complexes. Thus, the rapidly formed, initial enzyme-inhibitor (“fast-loose”) complexes demonstrate the relaxed specificity shown here in the nongentropic responses, whereas the slow-tight complexes have their parallel in the classically recognized sex-specific stable complexes needed for genotropic activity.

Consistent with the in vitro anti-apoptotic effects of sex steroids on osteoblasts and osteocytes, the loss of estrogens or androgens shortens the lifespan of osteoblasts and osteocytes in gonadectomized mice from either sex. An increase in osteocyte apoptosis following loss of estrogen has been demonstrated previously in rats as well as in humans (Noble,B. S. and Reeve,J. (2000) Mol. Cell Endocrinol. 159, 7-13), and contrasts with pro-apoptotic effects of estrogen on osteoclasts and the prolongation of their lifespan in estrogen deficiency (Hughes, D. E., et al. (1996) Nat. Med. 2, 1132-1136; Kameda,T., et al. (1997) J. Exp. Med. 186, 489-495). A shortened working life of the bone forming cell and longer working life of the bone resorbing cells could account, in part, for the imbalance between bone resorption and formation that follows loss of sex steroids. Moreover, an increase in osteocyte apoptosis could further weaken the skeleton by impairing the osteocyte/canalicular mechanosensory network (Manolagas, S. C. (2000) Endocr. Rev. 21, 115-137). Indeed, it seems likely that the signals of estrogens and those of mechanical strains may be integrated in bone, since, osteocytes which, like endothelial cells, are primarily regulated by shear stress and respond to estrogens, do also respond to stretching with ERK activation.

The dissociation of nongentropic from genotropic activities of the sex steroid receptors with synthetic ligands provide a novel means of separating desirable from undesirable actions of sex steroids, thereby providing improved pharmacotherapeutic agents. Specifically, the mechanism-specific ligands of the ERs or the AR, in distinction to tissue-specific ligands (SERMs) or classical estrogen or androgen, are an advantageous class of pharmacotherapeutic agents (true anabolic as opposed to anti-resorptive, and gender neutral) for the management of osteopenic states.

B.

Mice. Four-month-old female Swiss-Webster mice or five-month-old male SAMRI mice were maintained and used in accordance with NIH guidelines on the care and use of laboratory animals. Mice were subjected to a sham operation or to bilateral ovariectomy or orchidectomy. After 4 or 3 weeks, female or male mice, respectively, were sacrificed and the lumbar vertebrae removed.

Quantification of Apoptotic Osteoblasts and Osteocytes in Undecalcified Bone Sections

Apoptotic osteoblasts and osteocytes were detected in undecalcified plastic-embedded sections of the vertebrae (L1 to L5) by TUNEL staining using the Klenow FragEL detection kit (Oncogene), as previously described (Weinstein, R. S., et al. (1998) J. Clin. Invest. 102, 274-282.; Jilka, R. L., et al. (1999) J. Clin. Invest. 104, 439-446). TUNEL-positive hypertrophic chondrocytes were observed at the bottom of the growth plates serving as an internal positive control for each bone section.

Cell cultures. Osteoblastic cells were obtained from neonatal murine calvaria by collagenase digestion, cultured for 2-3 days in αMEM containing 10% FBS, resuspended using trypsin-EDTA, and frozen until use. Osteocytic MLO-Y4 cells (Kato,Y., et al. (1997). J. Bone Miner. Res. 12, 2014-2023) were cultured in phenol red-free αMEM supplemented with 2.5% FBS, 2.5% bovine calf serum, and cultured on collagen type I coated plates. Osteoblastic OB-6 cells (Lecka-Czemik, B. et al. (1999) J Cell Biochem. 74, 357-371) were cultured in phenol red-free αMEM supplemented with 5% FBS. HeLa cells were cultured in phenol red-free MEM supplemented with 10% FBS, 0.1 mM nonessential amino acids, and 1 mM sodium pyruvate.

Quantification of apoptotic cells in vitro. Apoptotic cells were quantified either by trypan blue staining or by direct visualization of changes in nuclear morphology (in the case of transfected HeLa cells), as previously described (Plotkin,L. et al. (1999). J.

Clin. Invest. 104, 1363-1374; Jilka,R. L. et al. (1998) J. Bone Miner. Res. 13, 793-802; Jilka, R. L. et al. (1999) J. Clin. Invest. 104, 439-446). In the latter method, the visualization of pyknotic or fragmented nuclei was facilitated by co-transfections of cells plated on glass coverslips with the enhanced fluorescent proteins (green or red) containing a nuclear localization sequence (EGFP-nuc or ERFP-nuc). Following fixation in 3.7% formaldehyde, the percentage of apoptosis was determined by determining the nuclear morphology in 200-500 transfected (fluorescent) cells.

Anoikis assay. Osteoblastic OB-6 cells (3.3×10⁵/ml) were cultured in type I collagen gels, essentially as previously described (Klein, C. E., et al. (1991) J. Cell Biol. 115, 1427-1436). After gels formed, they were detached from the bottom of the well using a spatula, and overlaid with 0.5 ml of (αMEM containing 10% FBS, 200 μM ascorbic acid, and either vehicle (0.1% ethanol) or steroids. After fixation, ten-micron sections were cut and apoptotic cells were quantified by TUNEL staining.

Western blot analysis. The phosphorylation status of ERK1/2 was analyzed by immunoblotting using a mouse monoclonal antibody recognizing tyrosine phosphorylated ERK1/2, or a rabbit polyclonal antibody recognizing total ERK1/2, followed by incubation with either an anti-mouse or an anti-rabbit antibody conjugated with horseradish peroxidase (Santa Cruz Biotechnology).

DNA constructs. Reporter plasmids carrying the 2.25 Kb of the human IL-6 promoter, 3 copies of the vitellogenin ERE sequences, or the complement 3 promoter driving the firefly luciferase gene (IL-6-luc, ERE-luc, and C3-luc, respectively) were used to assay transcriptional effects of the steroids. The expression plasmids of ER peptide antagonists carrying either αII, or two copies of 293 (2x293) , or two copies of F6 (2xF6) or GRIP fused to the GAL4 DNA binding domain driven by the SV40 promoter have been described previously (Norris, J. D. et al. (1999) Science 285, 744-746). The sequences of the peptides are disclosed in Chang et al. (1999) Mol. Cell. Bio. 19:8226-8239 which is incorporated herein in its entirety. The sequence for peptide αII is SSLTSRDFGSWYASR (SEQ ID NO.: 1). The sequence for peptide 293 is SSIKDFPNLISLLSR (SEQ ID NO.: 2). The sequence for peptide F6 is GHEPLTLLERLLMDDKQAV (SEQ ID NO.: 3). The sequence for GRIP is HSRLHDSKGQTKLLQLLTTKSDQMEPSPLPSSLSDTNKDSTGSLPGPGSTHGT SLKEKHKILHRLLQDSSSPVDLAKLTAEATGKELSQESSSTAPGSEVTVKQEP ASPKKKENALLRYLLDKDDTKDIGLPE (SEQ ID NO.: 4). Similarly, expression plasmids for the ERα mutants ΔA/B; S104,106,118A; ΔF; S554fs; ΔDBD; or L525A have been reported earlier (Schodin, D. J. et al. (1995) J Biol. Chem. 270, 31163-31171; Ekena, K., et al. (1996) J Biol. Chem. 271, 20053-20059; Montano, M. M., et al. (1995) Mol. Endocrinol. 9, 814-825; Montano, M. M. and Katzenellenbogen, B. S. (1997) Proc Natl. Acad. Sci. U.S.A. 94, 2581-2586; McInerney, E. M. and Katzenellenbogen, B. S. (1996) J Biol. Chem. 271, 24172-24178; Le Goff et al. (1994) J Biol. Chem. 269, 4458-4466; Wrenn, C. K. and Katzenellenbogen, B. S. (1993) J Biol. Chem. 268, 24089-24098; Ince, B. A. et al. (1993) J. Biol. Chem. 268, 14026-14032). For the construction of the DE and E mutants of the ERα, nucleotides corresponding to amino acids 263 through 553 and 333 through 553, respectively, were amplified from ERα by PCR. The forward primer contained the restriction site for SalI and the ERα Kozak sequence and the reverse primer contained the BamH1 restriction site. Amplified PCR products were subcloned into the SalI/BamH1 restriction site of pCMV.

The ERα-ECFP, E-ECFP, E-Nuc-ECFP and E-Mem-ECFP constructs were generated by PCR amplification of ERα using primer pairs 5′-GCCGCTAGCACCATGACCATGACCCTCCAC-3′ (SEQ ID NO.: 5) and 5′-GCCACCGGTCTGACTGTGGCAGGGAAACC-3′ (SEQ ID NO.: 6) or 5′-GCCGCTAGCACCATGAAGAACAGCCTGGCCTTG-3′ (SEQ ID NO.: 7) and 5′-GCCACCGGTCTAGTGGGCGCATGTAGGCG-3′ (SEQ ID NO.: 8), respectively. PCR products were digested with NheI and AgeI and inserted into the same sites in the appropriate ECFP vector. To generate E-Mem-ECFP, E was cloned into the Mem-ECFP vector (Clontech) which carries the N-terminal 20 amino acids of neuromodulin containing a signal for post-translational palmitoylation of cysteines 3 and 4 that target the fusion protein to the plasma membrane. To generate E-Nuc-ECFP, E was cloned into the Nuc-ECFP vector (Clontech) which carries three copies of the nuclear localization signal of the simian virus 40 large T-antigen fused to the C-terminus of ECFP. For the generation of ERα-ECFP and E-ECFP, the nuclear localization signal was eliminated from the Nuc-ECFP vector by Bglll/BamHI digestion and religation to generate ECFP. ERα or E PCR products were then ligated into the NheI/AgeI sites of ECFP. The complete sequence of all PCR-amplified regions was verified by sequencing. The cDNA for Src ASH3, Src ASH2, and SrcK295M (Src K⁻) were gifts from Dr. William C. Home, Yale University (Zhang, Z. et al. (2000) J. Biol.Chem 275:479-486). Wt or Shc mutants were provided by Dr K. S. Ravichandran, University of Virginia (Walk, S. F. et al. (1998) Eur. J. Immunol. 28, 2265-2275). dn MEK was provided by Dr N. G. Ahn, University of Colorado (Mansour, S. J. et al. (1994) Science 265, 966-970).

Transient transfections. HeLa cells were plated at 5×10⁴/cm² and after 16-24 hours were transfected with a total amount of 3 μg of DNA using Lipofectamine (Life Technologies Inc). Following a 4-6 hours of incubation, the transfection mix was replaced with fresh medium containing 10% charcoal-stripped serum and treatments were initiated. The IL-6 transcriptional analysis was performed as before (Pottratz, S. T. et al. (1994) J. Clin. Invest. 93, 944-950). For ERE- or C3-mediated transcription, transfected cells were treated with E₂ and luciferase and β-galactosidase activities were measured 24h later.

RT-PCR analysis of the ERs and AR in embryonic fibroblasts. The primers used were: for ERα (350 bp) 5′-TCTGCCAAGGAGACTCGCTACTGT-3′ (SEQ ID NO.: 9) and 5′-CTTGGCCAAAGGTTGGCAGC-3′ (SEQ ID NO.: 10); for ERβ (162 bp) 5′-GAAGTGCCAGCGAGCAGGTG-3′ (SEQ ID NO.: 11) and 5′-TGCTGGGACGGCTCACTAGCACAT-3′ (SEQ ID NO.: 12); for AR (615 bp) 5′-TGTGTGGAAATAGATGGG-3′ (SEQ ID NO.: 13) and 5′-TACATGTGGTCAAGTGGG-3′ (SEQ ID NO.: 14); and for β-actin (200 bp) 5′-TGGAGAAGAGCTATGAGCTGCCTG-3′ (SEQ ID NO.: 15) and 5′-GTGCCACCAGACAGCACTGTGTTG-3′ (SEQ ID NO.: 16).

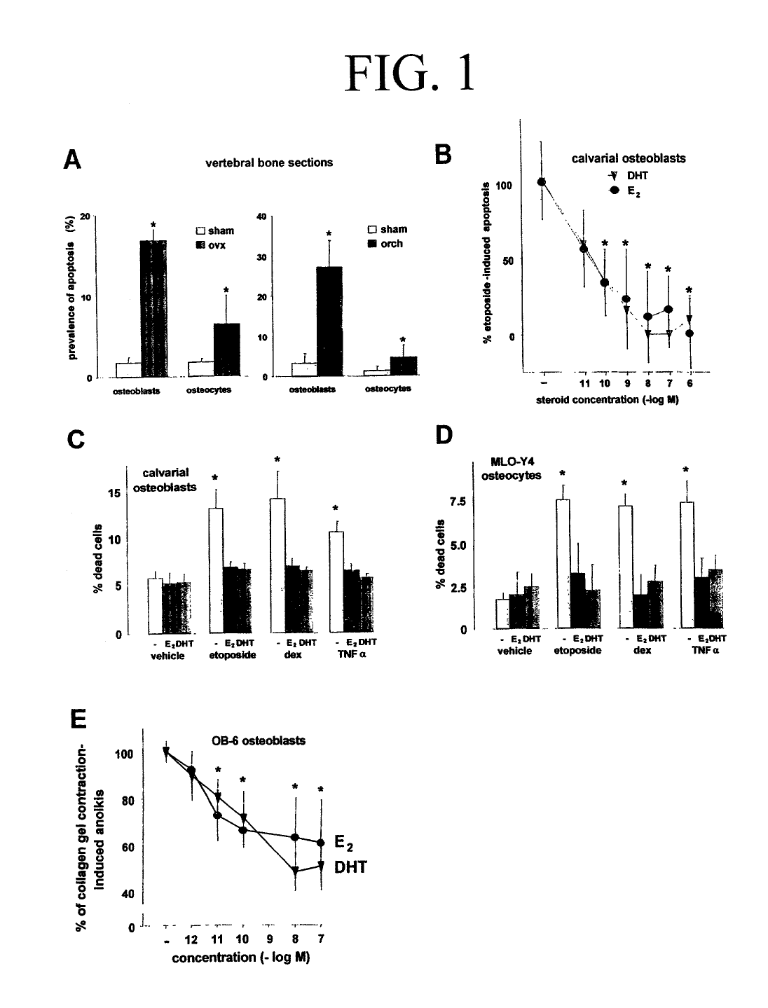

Statistical analysis. The Student's t test was used to evaluate the in vivo changes in bone cell apoptosis to assess for significant differences between means of the groups (n=4-5), after establishing equivalence of variances and normal distribution of data. The data of the in vitro apoptosis assays were analyzed by ANOVA, and the Student-Neuman-Keuls method was used to estimate the level of significance of differences between means. In order to establish whether the steroid effect was dependent on the pro-apoptotic agent used, data of FIGS. 1C and 1D were analyzed by two-way ANOVA, in which the two variables were the proapoptotic agents (vehicle, dexamethasone, etoposide, and TNFα) and the pretreatments (vehicle, E₂, and DHT). Subsequently, the Bonferroni method was used to estimate the significance of the differences among pretreatment-agent combinations.

### EXAMPLE 1

### Regulation of Osteoblast and Osteocvte Apoptosis by Estrogen or Androgen in vivo and in vitro

Ovariectomy or orchidectomy in mice increased vertebral osteoblast apoptosis by 10- and 8.3-fold and osteocyte apoptosis by 4- and 3.5-fold, respectively, compared to sham-operated controls (FIG. 1A). These changes were prevented in ovariectomized mice receiving 17β-estradiol (E₂).(data not shown). To elucidate the mechanism of these effects, primary clavaria cell cultures and an osteocytic cell line were used as in vitro models. Either E₂ or 5β-dihydrotestosterone (DHT) inhibited etoposide-induced apoptosis in calavaria cells in a dose-dependent fashion with a maximal effect seen at 10⁻⁹ to 10⁻⁸ M (FIG. 1B); identical results were obtained with MLO-Y4 cells (not shown). E₂ or DHT prevented apoptosis of clavaria-derived murine osteoblastic cells (FIG. 1C) or the osteocytic cell line MLO-Y4 (FIG. 1D) when induced by any of three different pro-apoptotic stimuli: etoposide, dexamethasone, or TNFα; as well as Fas-Fas ligand (not shown).

The signals that control the prevalence of apoptosis of osteoblasts and osteocytes in vivo are unknown. However, there is extensive evidence to suggest that interactions of cells with their matrix, in particular collagen-integrin interactions, are important in vivo cues for the decision of a cell to survive or initiate the process of suicide by apoptosis. This phenomenon has been termed anoikis (Frisch, S. M. and Ruoslahti, E. (1997) Curr. Opin. Cell Biol. 9, 701-706). Therefore the effects of sex steroids on anoikis using a bone marrow derived osteoblastic cell line (OB-6), which was cultured inside Type I collagen gels were studied (FIG. 1E). In this system, apoptosis results from loss of integrin signaling during contraction of the collagen lattice. As in the case of the other pro-apoptotic signals, either E₂ or DHT does-dependently attenuated anoikis at concentrations as low as 10⁻¹¹M.

### EXAMPLE 2

### ER (α or β) or the AR Transmit Anti-apoptotic Signals with the Same Efficiency Irrespective of Whether the Ligand is an Estrogen or an Androgen

The estrogen receptor antagonist ICI 182,780 or the androgen receptor antagonist flutamide blocked the anti-apoptotic effect of E₂ and DHT in calvaria cells, indicating strongly that the effects of the sex steroids were mediated by the ER and AR. Surprisingly, however, the anti-apoptotic effect of E₂ was also abrogated by flutamide, and the effect of DHT by ICI 182,780 (FIG. 2A). Consistent with this unexpected result, others have shown previously that anti-hormones including ICI 182,780 interact with multiple classes of steroid receptors (Nawaz, Z. (1999) Cancer Res. 59, 372-376).

To establish the dependency of the anti-apoptotic effect of E₂ and DHT on the ER and AR and dissect the role of each receptor in the unexpected results with the antagonists, studies with HeLa cells, which do not express ER or AR were performed. In these experiments, HeLa cells were transiently transfected with enhanced green fluorescent protein containing a nuclear localization sequence (EGFP-nuc) and either ERα, ERβ or the AR expression constructs, or empty vector controls (FIG. 2B). Neither E₂ nor DHT (10⁻¹²M to 10⁻⁸ M) had an effect on etoposide-induced apoptosis in cells transfected with the vector without receptor (empty vector, ev). However, in HeLa cells transfected with either the ER (α or β or the AR, but not the vitamin D receptor (VDR) or the retinoid X receptor (RXR), both E₂ or DHT prevented apoptosis in a dose-dependent manner over the same range of concentrations. Interestingly, 1,25(OH)₂D₃ prevented apoptosis in HeLa cells transfected with the VDR or RXR, but not the ERs or the AR. Identical results were obtained using dexamethasone to induce apoptosis.

As in the experiments with the primary cultures of osteoblastic cells, either ICI 182,780 or flutamide abrogated the effect of E₂ or DHT in HeLa cells, irrespective of whether the HeLa cells were transfected with the ERα or the AR (data not shown). These results clearly establish the requirement of the ER (α or β) or AR for the anti-apoptotic effects of estrogen. Moreover, the data suggests that the receptors responsible for nongentropic effects of sex steroids are indeed the classical receptor proteins, i.e. the ones that mediate the reproductive functions of estrogen or androgen, rather than alternative proteins. More important, these findings indicate that some actions of the classical ERs and AR are sex non-specific. Transcriptional regulation of the ER by androgen and the AR by estrogen have been noted recently in breast cancer cells (Maggiolini, M. et al. (1999) Cancer Res 59:4864-4869,), but this phenomenon could be explained by the expression of specific receptor coactivators in some cells (Yeh, S. et al. (1998) Pro Natl.Acad.Sci.U.S.A. 95:5527-5532,). The results presented herein represent the first demonstration that a non-transcriptional action of the classical ERs and AR is sex non-specific. Sex non-specific signaling by estrogen or androgen through the ER or the AR appears to account for the efficacy of either class of sex steroids in the adult skeleton of females and males and the equivocal skeletal phenotype of ER or aromatase deletions or mutations in rodents and humans.

### EXAMPLE 3

### The Anti-apoptotic Activity of the ERα is Located within the E (Ligand Binding) Domain and Requires Extra-nuclear Localization of the Protein

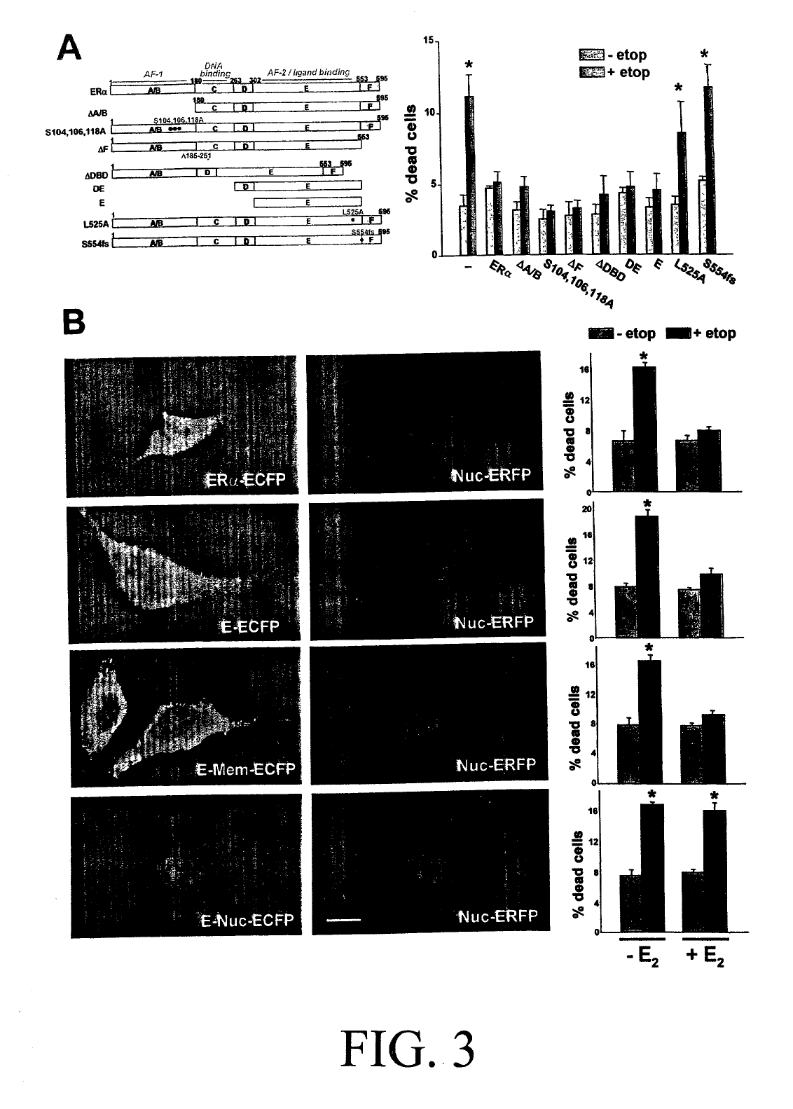

Using the ERα as a prototype, whether the anti-apoptotic activity of the receptors for sex steroids require the same or different domains than those required for transcriptional activity was determined (FIG. 3A). The effects of several mutants of the ERα on prevention of apoptosis were compared. Some of these mutants have been described previously and have been shown to produce the expected proteins when transiently transfected into human cells, such as the HeLa cells used in the present study (Schodin, D. J. et al. (1995) J Biol. Chem. 270, 31163-31171; Kraus, W. L. et al. (1997) J Steroid Biochem. Mol. Biol. 63, 175-188.; Ekena,K. et al. (1996) J Biol. Chem. 271, 20053-20059; Ekena, K., et al. (1997) J Biol. Chem. 272, 5069-5075; Montano et al., (1995) Mol. Endocrinol. 9, 814-825). In experiments not shown, it was established that the mutants comprising the DE or E domains also produced the expected proteins and lacked transcriptional activity.

Like ERα, mutant ΔA/B which lacks the entire N-terminal transcription activation function (AF-1) domain, or a mutant in which serines 104, 106, and 118 were substituted with alanine (S104,106,118A), or a mutant ΔF, which lacks the entire F domain of the carboxy terminus, or mutant ΔDBD which lacks part of the DNA binding domain (amino acids 185-251), were able to convey the anti-apoptotic signal of the E₂-bound ER. Yet, all these mutants exhibit reduced or no ERE-mediated gene transcriptional activity (Montano,M. M. and Katzenellenbogen,B. S. (1997) Proc Natl. Acad. Sci. U. S. A. 94, 2581-2586; Le Goff, P. et al. (1994) J Biol. Chem. 269, 4458-4466; McInerney, E. M. and Katzenellenbogen, B. S. (1996) J Biol. Chem. 271, 24172-24178; Montano et al., (1995) Mol. Endocrinol. 9, 814-825). More striking, the ability of the ligand to convey the anti-apoptotic signal was preserved in a deletion mutant which is missing the entire AF-1 and DNA binding domain (DE); as well as in a deletion mutant consisting solely of the AF-2/ligand binding domain (E). In contrast, the ER mutant L525A which lacks hormone binding ability (Ekena et al., (1997) J Biol. Chem. 272, 5069-5075) and the mutant S554fs which is deficient in the C-terminal transcription activation function (AF-2) (Schodin et al. (1995) J Biol. Chem. 270, 31163-31171; Ince et al. (1993) J. Biol. Chem. 268, 14026-14032; Wrenn and Katzenellenbogen (1993) J Biol. Chem. 268, 24089-24098), lost the ability to convey both the anti-apoptotic signal and the transcriptional activity of the protein (Ekena, K. et al. (1996) J Biol. Chem. 271, 20053-20059; Schodin et al., (1995) J Biol. Chem. 270, 31163-31171). These results indicate that the “anti-apoptotic” domain of the ER resides within the ligand binding region (domain E) of the protein and its anti-apoptotic activity may not require phosphorylation of certain N-terminal A/B domain sites important for the transcriptional activity of the ER.

Next whether the anti-apoptotic activity of the E domain was dependent on its localization to a particular subcellular compartment was determined (FIG. 3B). The E domain was targeted either to the plasma membrane or to the nucleus of HeLa cells, by fusing it to enhanced cyan fluorescent protein containing plasma membrane or nuclear localization sequences, respectively. As revealed by epifluorescence microscopy, the non-targeted ERα and the non-targeted E exhibited similar distribution of cyan fluorescence in the nucleus of the cytoplasm, indicating that elimination of all but the ligand binding domain of the ER does not alter the subcellular distribution of the protein. However, incorporation of the appropriate targeting sequence accomplished the expected subcellular localization of the membrane or the nuclear ECFP fusion proteins, respectively, as can be seen by comparing and contrasting the distribution of cyan and red fluorescence in the same cell(s). Targeting the E domain predominantly to the plasma membrane did not alter its ability to convey the ligand-induced anti-apoptotic signal, as evidenced by identical anti-apoptotic activity of this fusion protein to that of the E domain or the full length ERα . But in sharp contrast, targeting the E domain exclusively to the cell nucleus resulted in complete loss of its anti-apoptotic activity.

### EXAMPLE 4

### The Anti-apoptotic Activity of the ER and R is Mediated via a Src/Shc/ERK Signaling Pathway

E₂ or DHT rapidly and transiently increased the phosphorylation of ERKs in MLO-Y4 cells, with a maximal effect seen at 5 min and a return to baseline by 15 min (FIG. 4A). Identical results were reproduced using the murine calvaria osteoblastic cells (not shown). The induction of ERK phosphorylation by E₂ or DHT was blocked by the specific inhibitors of the phosphorylation of ERKs, PD9809 and UO 126 (not shown); as well as the Src family tyrosine kinase inhibitor PP1. Moreover, both PD98059 and PP1 completely abrogated the anti-apoptotic effect of E₂ and DHT, indicating that ERK phosphorylation and Src kinase activity are require for the anti-apoptotic effects of both sex steroids. Confirmatory evidence for the essential role of Src knase in the anti-apoptotic effects of sex steroid was provided by the finding ER- and AR-expressing embryonic fibroblasts derived from Src−/− mice that also lack yes and Fyn (Sorinao et al. (1991) Cell 64:693-702), unlike the Src+/+ wild type controls, were unresponsive to the anti-apoptotic effect of E₂ or DHT (FIG. 4B).

The co-dependency of the anti-apoptotic effect of E₂ and DHT on the sex steroid receptors (either the ER or AR) as well as on the activation of Src/ERK signaling pathway was demonstrated in HeLa cells (FIGS. 4C and D). PD98059 or co-transfection of a dominant negative (dn) MEK, a kinase responsible for the phosphorylation of ERKs, abrogated the effects of E₂ or DHT in HeLa cells transfected with ERα or AR (FIG. 4C). Likewise, the effect of E₂ or DHT was abrogated in HeLa cells transfected with the ERα or AR and co-transfected with a Src mutant lacking the kinase activity (Src K) (FIG. 4D). More strikingly, a Src mutant lacking the SH2 domain (Src ASH2) abrogated the anti-apoptotic activity of ligand-activated ERα but not that of the AR. Conversely, a Src mutant lacking the SH3 domain (Src ASH3) abrogated the anti-apoptotic activity of the AR but not of the ER. Likewise, the anti-apoptotic activity of the E₂ or DHT activated ERα was abrogated by dominant negative Shc mutants in which al three, or only the third of the three tyrosines (Y239, Y240, and Y317) which are the primary sites of Shc phosphorylation were substituted by phenylalanine. However, when tyrosines 239 and 240, but not 317, were mutated the anti-apoptotic activity of the receptor was unaffected (FIG. 4E).

Consistent with the evidence that the E domain of the ER is sufficient for its anti-apoptotic activity (FIG. 3), in HeLa cells transfected with this domain, PD098059, the dn MEK, the Src K; Src ASH2 or Src ASH3 had exactly the same effects as they did in HeLa cells transfected with the full length ERα (FIG. 4C and D).

### EXAMPLE 5

### Dissociation of the Transcriptional from the Anti-apoptotic Activity of the ERα using Peptide Antagonists

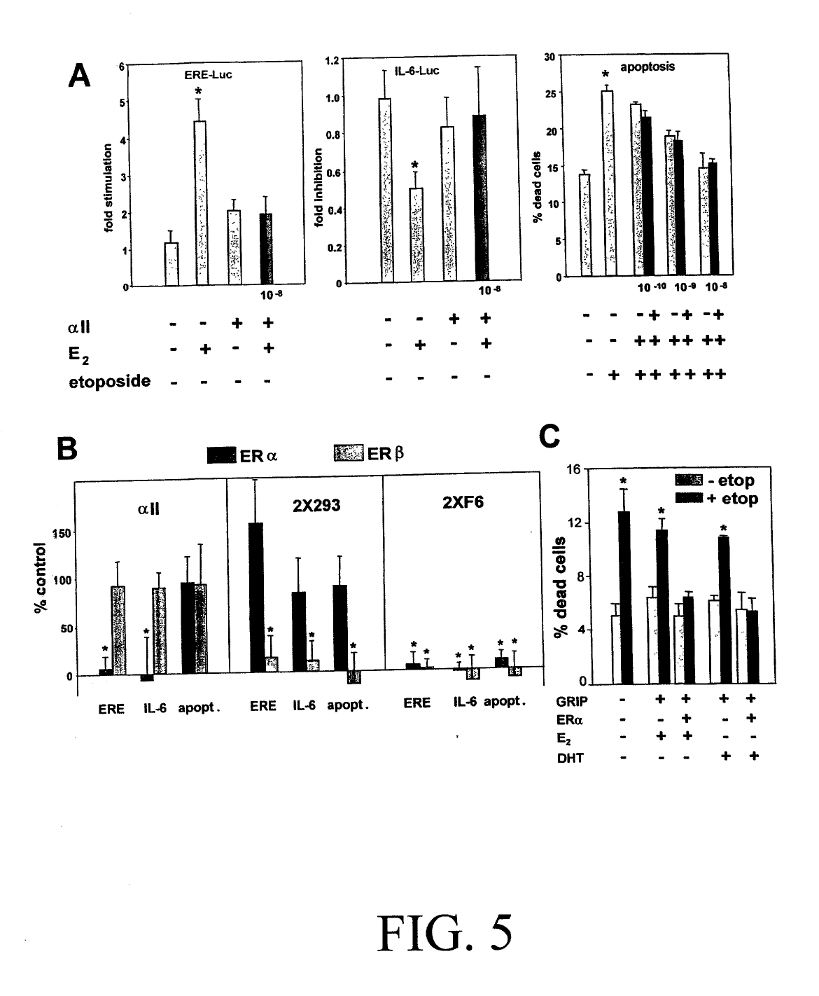

McDonnell and co-workers have previously isolated several classes of small (11-19 amino acid) peptides that bind district site of the ligand-activated ER, and can selectively block ERα but not ERβ-mediated transcription, and vice versa, when tested on a consensus ERE (estrogen response element) (Norris et al. (1999) Science 285:744-746; Chang et al. (1999) Mol. Cell Biol. 19:8226-8239). The ability of these peptides to block the anti-apoptotic effects of E₂ was examined (FIG. 5). In parallel experiments, the ability of these peptides to block transcription was examined using HeLa cells transfected with an ERE driven luciferase construct (ERE-Luc) or an IL-6 promoter-driven luciferase (IL-6-Luc). The peptide designated αII, having sequence SSLTSRDFGSWYASR (SEQ ID NO.: 1) which binds to the ligand binding domain ERα, blocked the E₂-included increase in ERE-Luc transcription (FIG. 5A). Likewise, the peptide blocked an E₂ -induced decrease in the transcription of IL-6-a paradigm of transcriptional regulation mediated via protein-protein interaction between the ER and other transcription factors. Nonetheless, the αII peptide did not influence the anti-apoptotic effect of E₂. Identical results were obtained in experiments using HEK293, instead of HeLa cells (not shown).

An ERβ-specific peptide (293), and a peptide which interacts with both the ERα and ERβ (F6) (FIG. 5B) were investigated. For these experiments, HeLa cells were transfected with the ERE- or IL-6 luciferase constructs and either the ERα or the ERβ. 293 blocked the effect of E₂ on transcription from both the ERE and the IL-6 promoters in the ERβ carrying cells, but not in the ERα carrying cells. F6, on the other hand, blocked the effect of E₂ on transcription from ERE or IL-6 in both ERα and ERβ carrying cells. More importantly, unlike αII, which did not affect the anti-apoptotic activity of ERα (and as expected of ERβ, as αII is specific for ERα), 293 did block the anti-apoptotic activity of the ERβ, but it did not have an effect on the anti-apoptotic activity of the ERα. F6 blocked the anti-apoptotic activity of both ERα and ERβ, as well as transcription from ERE and IL-6. These results confirm the evidence that each peptide binds to and blocks different sites of the AF-2 binding pocket (Norris, J. D. et al. (1999) Science 285:744-746; Chang, C. et al. (1999) Mol.Cell Biol. 19:8226-8239). Moreover, the data also indicates that blocking the site to which αII binds, and only this site, does not affect the “anti-apoptotic” activity, even though it effectively blockades the transcriptional activity of the ERα. The demonstration of the specificity of these peptides in blocking transcriptional or/and anti-apoptotic activity from the ERα or ERβ supports the contention that the anti-apoptotic activity of the ligand activated receptor depends on a distinct site of the region of the receptor protein, which has been conveniently termed “anti-apoptotic” domain.

The transcriptional activity of the ER is dramatically influenced by its interaction with specific co-activators, most of which contain the conserved LXXLL motif termed the nuclear receptor box (McKenna et al. (1999) Endocr. Rev. 20, 321-344). Peptide GRIP prevents the interaction of the ER with GRIP-1, one of these co-activators (Chang, C. et al. (1999) Mol.Cell Biol. 19:8226-8239). The GRIP peptide contains the center three copies of the LXXLL motifs from the co-activator GRIP-1. The nucleotide sequence for that region is:

| | |
|---|---|
| CAC AGCCGGCTGC ATGACAGCAA AGGGCAGACC AAACTCCTGC | (SEQ ID NO:17). |

| | | | |
|---|---|---|---|
| AGCTGCTGAC | CACCAAGTCC | GACCAGATGG | AGCCTTCACC |
| CTTGCCCAGC | TCCTTGTCGG | ACACAAACAA | GGACTCAACA |
| GGGAGCTTGC | CTGGGCCTGG | GTCCACGCAT | GGCACCTCGC |
| TCAAGGAGAA | GCATAAGATT | TTGCACAGAC | TCTTACAGGA |
| CAGCAGTTCC | CCTGTGGACT | TGGCCAAGCT | GACAGCAGAA |
| GCCACAGGCA | AAGAGCTGAG | CCAGGAGTCC | AGCAGCACAG |
| CTCCTGGGTC | GGAAGTGACT | GTCAAACAGG | AGCCAGCGAG |
| CCCCAAGAAG | AAAGAGAATG | CACTACTGCG | CTATTTGCTC |

GACAAAGATG ATACTAAAGA TATTGGTTTA CCGGAA

When this peptide was co-transfected with ERα into HeLa cells, it did not influence the anti-apoptotic activity of the ER (FIG. 5C), arguing further that the anti-apoptotic activity of the ligand-activated receptor is independent of the function of this protein as a nuclear transcription factor.

### EXAMPLE 6

### The Transcriptional Activity of the ERα can be Dissociated from its Nongentropic Activity with Synthetic Ligands

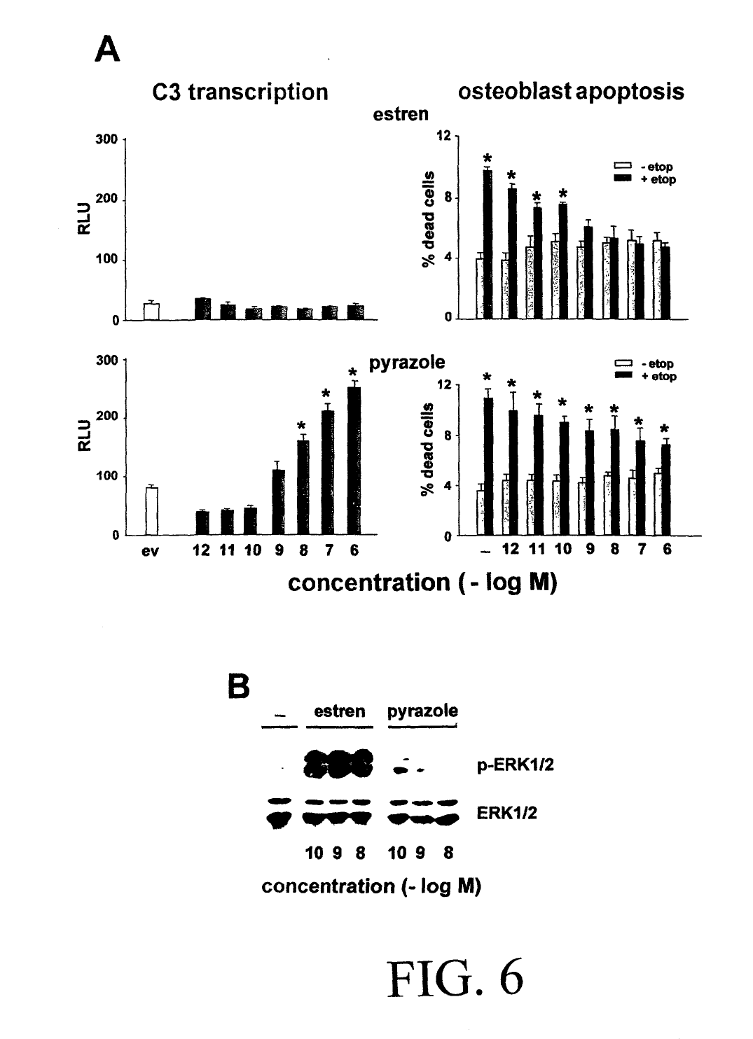

A comparison of the effects of various ligands on the transcription of the C3 gene, which contains an ERE in its promoter and is regulated by estrogen in a classical manner, with their anti-apoptotic effect on primary calvaria cells was conducted. As depicted in FIG. 6A, an estren was found that had no transcriptional activity, exhibited potent anti-apoptotic efficacy. On the other hand, a pyrazole with potent transcriptional activity, had minimal anti-apoptotic efficacy. The relative anti-apoptotic potency of these two compounds correlated with their ability to induce ERK phosphorylation (FIG. 6B).

### EXAMPLE 7

### Transgenic Animals using the Peptide F6 as the Transgene

To dissect in vivo the relative contribution of the actions of the ERs versus the actions of the AR to the bone protective effects of estrogen or androgen can be accomplished by comparing osteoblastogenesis and osteoclastogenesis, osteoblast and osteoclast apoptosis, bone mass and bone strength in wild type ovariectomized mice and mice carrying a tetracycline-inducible transgene encoding the ERs-peptide antagonist 2XF6. Estrogen can exert anti-apoptotic effects as effectively and with identical potency through the ERα or ERβ or AR. Additionally, the ERα or ERβ-mediated effects can be blocked using the selective ER peptide antagonist 2XF6 (FIG. 5B). The activity of estrogen or androgen through the AR can be retained while simultaneously blocking the activity of these steroids through ERα and ERβ by using 2XF6 as the transgene. Developmental problems like the ones that are most likely confounding the results of the ERKO mice, can be avoided by placing the transgene under the control of the TET ON system driven by the actin promoter and as described in Gossen, M., et al. (1993) Trends.Biochem.Sci. 18:471-475, and Gossen, M. et al. (1995) Science 268:1766-1769.

The reverse tetracycline repressor (rTetR or TET-ON) system takes advantage of the high specificity of the TetR-operator-Tc interaction, the herpes simplex virus (HSV)-VP16 transcription activation domain, and the tetracycline (Tc). In the convention (TET-OFF) system, the Tc-controlled transactivator (tTA, a fusion between TetR and the activating domain of the VP16 protein) stimulated the gene expression, as has been shown with reporter assays in HeLa cells. The efficiency of stimulation was proven by a 10⁵-fold increase in the reporter gene expression levels. Tc can prevent tTA from binding to tet operators (tetO) upstream of a minimal promoter, and consequently turns off tTA-dependent expression. However, the TET-ON system utilizes a rtTA transactivator that has been modified such as Tc, or its derivative Doxycycline (Dox) induces rather than abolishes binding of VP16 to the operator. The TET-ON system circumvents any potential toxic effects that may be conferred to eukaryotic cells following prolonged exposure to tetracycline.

Two constructs are introduced into the same mouse for Dox-induced expression of the peptides. In the first construct, the gene encoding rtTA (composed of rTETR and VP16 activation domain) is placed under the control of the actin promoter for ubiquitous expression. This promoter has been successfully used to direct ubiquitous expression of the human placental alkaline phosphatase gene in transgenic mice (DePrimo, S. E. et al. (1996) Transgenic.Res. 5:459-466). A human Growth Hormone (hGH) first intronic region with its nuclear localization signal (NLS) and polyadenylation site (polyA) is cloned downstream of the actin-rtTA construct to confer transcript stability and optimal protein expression levels. In the second construct, a tetracycline response element (TRE) that carries the tetO sequences and a minimal promoter, is placed upstream of a GAL4DBD2XF6-hGh-poly (a) cassette.

Two copies of the 2XF6 peptide are used in the GALA DNA binding domain-2XF6 peptide fusion because they are more efficient than just a single copy in suppressing both the transcriptional and anti-apoptotic activity of the ERs. Transgenic mice carrying the co-integrated constructs are grown to adulthood and then treated with Dox in the drinking water. In the absence of Doxycycline (DOX) the transactivator in the first construct is blocked from binding to the tetO sequences. Once Doxycycline is added the transactivator will recognize its target tetO sequences in the TRE and under the control of the actin promoter will drive transcriptional activation of the peptide sequences.

### Generation of DNA Constructs for Microinjection

The actin-rtTA- poly (A) and TREGAL4DBD2XF6-hGH-poly(A) DNA constructs are generated following standard subcloning techniques. Each one of the two constructs requires multiple steps of cloning. Every step of the final constructs is verified with sequence analysis and their function established in transient transfectants of HeLa cells by monitoring their ability to block the activity of both ERs in response to Dox treatment.

### Generation and Identification of Transgenic Mice

The actin-rtTA- poly (A) and TREGAL4DBD2XF6-hGh-poly (A) DNA constructs are excised from their respective vectors and are microinjected into fertilized mouse eggs. Eggs are allowed to reach the two-cell stage and reimplanted into the oviducts of pseudopregnant foster mice. The derived litters are screened by tail DNA PCR for co-integration of the actin-rtTA-poly(A) and the TRE-GAL4DBD2XF6-hGh-poly(A) transgenes. To investigate whether the two DNA cassettes have integrated in the same or similar location of the host genome, founders are mated with C57BL6 mice and the offspring screened for transgene transmission. Transgenic litter positive for both constructs, after tail DNA screening with PCR as described above, is selected for further characterization. The DNA sequence integrity of each integrated transgene is confirmed by Southern blot analysis of tail genotropic DNA and hybridization against probes homologous to the 5′ or the 3′ end of each transgene.

### Characterization of Transgene Expression in Mice

Identified transgenic animals (referred to as Tet-F6) are characterized in terms of their ability to switch on F6 peptide expression in response to Dox. Dual transgenic litter is maintained on normal water or water supplemented with 0.5 mg/ml Dpox., as it has been reported in the literature (Ray, P. et al. (1997) J Clin.Invest. 100:2501-2511), for varying periods of time. This approach evaluates the minimum amount of time required for the mice to be treated with Dox in order to inducibly express the F6 peptide. Mice are sacrificed before and at 2,4,6 days after the addition of Dox and tissues harvested. Total mRNA is extracted from calvaria, femur, vertebrae, uterus, mammary gland, and a number of soft tissues such as liver, lung, kidney, spleen, brain and F6 peptide expression are evaluated with Northern blot analysis. The same approach is followed to confirm that removal of Dox from the drinking water of the double transgenic animals results in cessation of peptide production.

### Evaluation of the Ability of the Peptides to Block Estrogen-induced Transcription

Double transgenic mice are crossed with ERE-lacZ transgenic mice. When the triple transgenic litter reaches adulthood Dox is added to the drinking water to induce and maintain peptide expression for at least 48 hours. To confirm that ER-mediated transcription has been blocked, animals are sacrificed following 24 hours treatment with 20 ng/g body weight of E₂ and LacZ activity are measured in the uterus, mammary gland, femur and vertebrae. Once peptide expression and blockade of ER transcription in the ERE-lacZ transgenic mice is confirmed, direct confirmation of the blocked is determined using WT mice and Tet-F6 mice induced to express F6 in adulthood for at least 28 days in either sham operated or ovariectomized mice. After surgery all groups are treated with E₂ for 28 days at doses of 20 ng/g body weight. Following the 28 day treatment period, mice are sacrificed and uteria and uteri and mammary glands isolated. Total RNA is extracted from these tissues and screened for the expression of lactoferrin, a gene that is transcriptionally regulated by estrogen via classical ERE binding. It is known that in WT mice, lactoferrin mRNA increases approximately 5-fold in the uterus, under these same experimental conditions. The functional efficacy of the ER peptide antagonist is established directly by the lack of lactoferrin expression in the Tet-F6 animals, but not in the overiectomized WT controls receiving estradiol.

### Analysis of the Skeletal Phenotype

The transgene is turned on for a period of 4-6 weeks in female Tet-F6 mice four month of age treated with either E₂ (20ng/g body weight) or DHT (20ng/g body weight) or vehicle. The skeletal phenotype of vehicle-or steroid-treated Tet-F6 mice is compared to that of wild type animals overiectomized at four months of age and maintained for another 4-6 weeks post-overiectomy without or with E₂ or DHT treatment. Tet-F6 and wild type animals are assessed for bone mass by in vivo DEXA analysis at weekly intervals following the turning on of the transgene or the overiectomy. At the end of the 4-6 week period, both groups of animals are sacrificed and the following determinations made: a) whole body and uterine and mammary gland weights, b) length of the long bones and the width between the periosteal and endosteal bone, c) osteoblastogenesis and osteoclastogenesis in ex vivo cultures of bone marrow cells, d) prevalence of osteoblast/osteocyte and osteoclast apoptosis in sections of vertebrae, e) rate of bone formation and resorption by dynamic histomorphometry and f) compression strength of the vertebrae. An intraperitoneal injection of 30ng/g body weight of oxytetracycline-hydrochloride is administered 8 days and 2 days before sacrificing the animals for the dynamic histomorphometry analysis. It is important to note that the TET ON system is 100-1000 fold less sensitive to tetracycline than it is to doxycycline. In addition, it is established that administration of doxycycline (0.5 mg/ml in drinking water) has no effect on bone remodeling and does not interfere with visualization of tetracycline labels formed in cancellous bone after injection (s.c) of 0.5 mg tetracycline. Therefore, this maneuver should not influence the expression of the transgene, which at this stage will have been turned on with doxycycline.

### EXAMPLE 8

### Transgenic Animals using αII and 293 as Transgenes

Transgenic mice carrying a tetracycline-inducible transgene encoding two ER peptide antagonists, αII+2X293 (from here onward referred to as Tet-peptides) can be generated using similar methods. Using αII +2X293 as the transgenes nongentropic activity through the ERα can be retained but transcriptional activity through both ERα and ERβ and the nongentropic activity of ERβ can be blocked. The 293 peptide blocks the transcriptional and non-transcriptional activity of ERβ , but has no effect on either the transcriptional or non-transcriptional activity of ERα

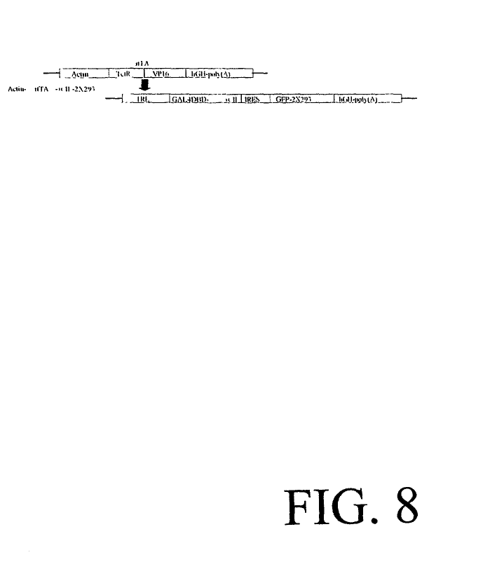

Two constructs are built and co-introduced into the same mouse for Dox-induced expression of the peptides (FIG. 8). In the first construct, the gene encoding rtTA (composed of rTetR and VP16 activation domain) is placed under the control of the actin promoter for ubiquitous expression. A human Growth Hormone (hGH) first intronic region with its nuclear localization signal (NLS) and polyadenylation site (poly(A)) is cloned downstream of the actin-rtTA construct to confer transcript stability and optimal protein expression levels. In the second construct, a tetracycline response element (TRE) that carries the tetO sequences and a minimal promoter, is placed upstream of a GAL4DBDαII-IRES-GFP2X293-hGh-poly(A) cassette. In this cassette, the GAL4 DNA binding domain-αII peptide fusion will be linked with an internal ribosome entry site (IRES, which permits the translation of two open reading frames from one mRNA) to two copies of the 293 peptide that will be fused to the green fluorescent protein gene (GFP).

The reason for using two copies of the 293 peptide is that preliminary experiments have indicated that they are more efficient than just a single copy in suppressing both the transcriptional and anti-apoptotic activity of the ERβ. Each of the αII and 2X293 peptides are fused to a different protein instead of the same GAL4DBD to avoid the possibility of recombination events occurring between the two identical GAL4DBD regions. Furthermore, the GFP is incorporated in the GAL4DBDαII-IRES-GFP2X293-hGh-poly(A) cassette to provide an easy means of screening for peptide expression. Transgenic mice carrying the co-integrated constructs are grown to adulthood and then treated with Dox in the drinking water. In the absence of Dox the transactivator in the first construct is blocked from binding to the tetO sequences. Once Dox is added, the transactivator can recognize its target tetO sequences in the TRE and, under the control of the actin promoter, will drive transcriptional activation of the peptide sequences.

Generation of DNA Constructs for Microinjection: The actin-rtTA- poly(A) and TRE-GAL4DBDαII-IRES-GFP2X293-hGh-poly(A) DNA constructs are generated following standard subcloning techniques. The integrity of the final constructs are verified with sequence analysis and function will be established in transient transfectants of HeLa cells by monitoring its ability to block the transcriptional activity of both ERα and ERα and the anti-apoptotic activity of ERβ.

Generation and Identification of Transgenic Mice: The actin-rtTA-poly(A) and TRE-GAL4DBDαII-IRES-GFP2X293-hGh-poly(A) DNA constructs are excised from their respective vectors and microinjected into fertilized mouse eggs. Eggs are allowed to reach the two-cell stage and are re-implanted into the oviducts of pseudopregnant foster mice. Tail DNA is screened by PCR for co-integration of the actin-rtTA-poly(A) and the TRE-GAL4DBDαII-IRES-GFP2X293-hGh-poly(A) transgenes. To investigate whether the two DNA cassettes have integrated in the same or a similar location of the host genome, founders are mated with C57BL/6 mice and the offspring will be screened for transgene transmission. Transgenic litter that will prove positive for both constructs, after tail DNA screening with PCR as described above, are selected for further characterization. The DNA sequence integrity of each integrated transgene is confirmed by Southern blot analysis of tail genotropic DNA and hybridization against probes homologous to the 5′ or the 3′ end of each transgene.

Characterization of Transgene Expression in Mice: Identified transgenic animals are characterized in terms of their ability to switch on αII and 293 peptide expression in response to Dox. Dual transgenic litters are maintained on normal water or water supplemented with 0.5 mg/ml Dox, for varying periods of time. This approach evaluates the minimum amount of time that is required for the mice to be treated with Dox in order to induce expression of the αII and 293 peptides. Mice are sacrificed before and at 2, 4, and 6 days after the addition of Dox, and tissues are harvested. Total mRNA is extracted from calvaria, femur, vertebrae, uterus, mammary gland, and a number of soft tissues such as liver, lung, kidney, spleen, brain, and αII and 293 peptide expression is evaluated with Northern blot analysis. The same approach will be followed in order to confirm that removal of Dox from the drinking water of the double transgenic animals will result in cessation of peptide production.

Evaluation of the Ability of the Peptides to Block Estrogen-Induced Transcription: To determine the selective inhibition of estrogenic transcriptional activity in the Tet peptide animals, two approaches are followed. First, double transgenic mice are crossed with ERE-lacZ transgenic. When the triple transgenic litter reach adulthood, Dox is added to the drinking water to induce and maintain peptide expression for at least 48 hours. To confirm that ER-mediated transcription has been blocked, animals are sacrificed following 24 hours treatment with 20 ng/g body weight of E₂, and lacZ activity is measured in the uterus, mammary gland, femur and vertebrae. For direct confirmation of the blockade of ER-mediated transcription. WT mice and Tet-peptides mice induced to express the peptides in adulthood for at least 28 days are either sham-operated or ovariectomized. After surgery, all groups are treated with E₂ for 28 days at doses of 20 ng/g body weight. Following the 28-day treatment period, mice are sacrificed and uteri and mammary glands is isolated. Total RNA is extracted from these tissues and screened for the expression of lactoferrin, a gene that is transcriptionally regulated by estrogens via classical ERE binding. Lack of lactoferrin expression in the Tet-peptides animals, but not in the ovariectomized WT controls receiving estradiol, establishes directly the functional efficacy of the ER peptide antagonists.

Analysis of the Skeletal Phenotype: Once the Tet-peptides mice are established and characterized as described above, females are grown to 4 months of age. At this time, the transgene is turned on for a period of 4-6 weeks and the skeletal phenotype will be compared with wild type animals ovariectomized at 4 months of age and maintained for another 4-6 weeks post-ovariectomy. At weekly intervals following the turning on of the transgene or the ovariectomy of the wild type mice, both animals are assessed for bone mass by in vivo DEXA analysis. The animals are sacrificed when the percent change in spinal BMD is at least −3% for the ovariectomized wild type group, or 6 weeks, whichever comes first. This difference requires 8 animals per group to exceed 80% power. At the end of the 4-6 week period, both groups of animals are sacrificed and the following determinations are made: a) whole body and uterine and mammary gland weights, b) length of the long bones and the width between the periosteal and endosteal bone, c) osteoblastogenesis and osteoclastogenesis in ex vivo cultures of bone marrow cells, d) prevalence of osteoblast/osteocyte and osteoclast apoptosis in sections of vertebrae, e) rate of bone formation and resorption by dynamic histomorphometry and f) compression strength of the vertebrae. An intraperitoneal injection of 30 μg/g body weight of oxytetracycline-HCl is administered 8 days and 2 days before sacrificing the animals for the dynamic histomorphometry analysis. It is important to note that the TET ON system is 100-1000 fold less sensitive to tetracycline than it is to Dox. In addition, administration of Dox (0.5 mg/ml in drinking water) has no effect on bone remodeling and does not interfere with visualization of tetracycline labels formed in cancellous bone after injection (s.c) of 0.5 mg tetracycline.

### EXAMPLE 9

### Transgenic Animals with Inducible Inactivated Sex Steroid Receptors

A true and informative model of postmenopausal or postcastration requires deleting each one of the sex steroid receptors, both in a cell specific manner (osteoblasts, osteoclasts and both) as well as ubiquitously. The Cre/loxP binary recombination system of bacteriophage P1 hour has been shown to efficiently mediate loxP site-specific recombination in both ES cells (Zou, Y. R. et al. (1994) Curr.Biol. 4:1099-1103; Gu, H. et al. (1993) Cell 73:1155-1164) and transgenic mice (Lakso, M. et al. (1992) Proc Natl.Acad.Sci.U.S.A. 89:6232-6236; Orban, P.C. et al. (1992) Proc Natl.Acad.Sci.U.S.A. 89:6861-6865).

### Generation of DNA Constructs for Microinjection

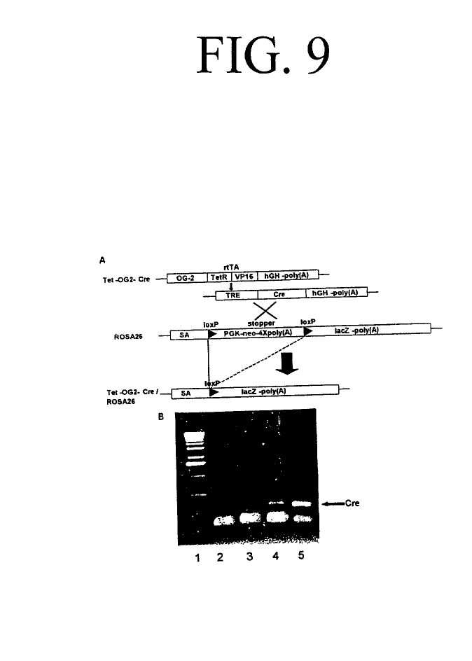

Conditional expression is achieved with the reverse tetracycline repressor (rTetR or TET-ON) system. In the first construct, the gene encoding rtTA was placed under the control of the osteocalcin (OG-2) promoter for osteoblast-specific expression. A human Growth Hormone (hGH) first intronic region with its nuclear localization signal (NLS) and polyadenylation site (poly(A)) was cloned downstream of the OG-2-rtTA construct to confer transcript stability and optimal protein expression levels. In the second construct, a tetracycline response element (TRE) was placed upstream of a Cre-hGh-poly(A) cassette. Each one of the two constructs required multiple steps of cloning (FIG. 9A). Every step was checked with appropriate restriction enzyme digestions and PCR reactions and the final constructs were sequenced. Furthermore, the specificity and efficiency of the Dox-induced Cre expression, was confirmed in an in vitro system using the Ob-6 osteoblastic cell line. These cells have been characterized in and were found to express osteocalcin and to express both the ERα and the ERβ. Ob-6 cells were transiently transfected with either the OG-2-rtTA-poly(A) and TRE-Cre-hGh-poly(A) constructs or the empty vectors and treated them with 1.5μg/ml Dox or with vehicle for 48 hours. Cells were harvested and used to extract total RNA. Semiquantitative RT-PCR analysis using Cre-specific primers indicated specific expression of Cre only in cell populations that had been treated with Dox (FIG. 9B).

### Generation and Identification of Transgenic Mice

The OG-2-rtTA- poly(A) and TRE-Cre-hGh-poly(A) DNA constructs were exercised from their respective vectors with the appropriate restriction endonucleases and were microinjected into the pronuclei of fertilized C57B1/6 mouse eggs. Eggs are allowed to reach the two-cell state and are reimplanted into the oviducts of pseudopregnant foster mice. Founders are identified by screening them for co-integration of the OG-2-rtTA and TRE-Cre transgenes. Mice transgenic for both the OG-2-rtTA and the TRE-Cre constructs (from here onwards referred to as Tet-OG2-Cre) are identified by PCR analysis of tail DNA with two sets of primers each of which has been designed to amplify part of the OG-2-rt-TA and the Cre DNA sequences. To investigate whether the two DNA cassettes have integrated in the same site of the mouse genome, founders are mated with C57B1/6 mice and the offspring are screened for transgene transmission. Transgenic litter positive for both constructs, after tail DNA screening with PCR as described above, are selected for further characterization. The DNA sequence integrity of each integrated transgene is confirmed by Southern blot analysis of tail genotropic DNA and hybridization against probes homologous to the 5′ or the 3′ end of each transgene.

### Functional Characterization of the Transgene

Identified transgenic animals are characterized in terms of their ability to switch on Cre expression in response to Dox. The dual transgenic litter is maintained on normal water or water supplemented with 0.5 mg/ml Dox, as it has been reported in the literature (Ray, P. et al. (1997) J Clin.Invest. 100:2501-2511), for varying periods of time. This approach evaluates the minimum required amount of time that is required for the mice to be treated with Dox. In order to confirm inducible expression of Cre, mice are sacrificed before and at 2, 4, 6 days after the addition of Dox and tissues are harvested. Total mRNA is extracted from calvaria, femur, vertebrae, uterus, mammary gland, and a number of soft tissues such as liver, lung, kidney, spleen, and brain and Cre expression is evaluated with Northern blot analysis. The same approach is followed in order to confirm that removal of Dox from the drinking water of the double transgenic animals results in cessation of Cre production.

To monitor Cre-mediated recombination Tet-OG2-Cre mice are crossed with the ROSA26 reporter mouse (Mao, X. et al. (1999) Proc Natl.Acad.Sci.U.S.A. 96:5037-5042) that is now available from Jackson Laboratories. In these mice, a floxed stopper fragment and the β-galactosidase-neomycin phosphotransferase fusion gene (βgeo) have been integrated into the ROSA26 locus. After Cre-mediated excision of the stopper fragment, expression of βgeo is driven by exon 1 of the ROSA26 allele and is ubiquitous during embryonic development and adulthood. Interbreeding the ROSA26 mouse with the functional Tet-OG2-Cre transgenic lines result in β-galactosidase expression in osteoblastic cells. Tissues such as calvaria, femur, and vertebrae are stained for β-galactosidase expression. A similar approach has been used to evaluate T-cell specific expression (Mao, X. et al. (1999) Proc Natl.Acad.Sci.U.S.A. 96:5037-5042).

### A Mutant Mouse Strain which Carries the Floxed Exon 3 of ERα

The second mouse strain (Ind-αERKO) carries exon 3 (653 -845bp) which contains 96 kb of the 0.249 Kb mouse ERα DNA binding domain (740-989 bp) (White, R., et al. (1987) Mol.Endocrinol. 1:735-744) flanked by two loxP sites, each of which consists of a series of 34 bp of tandem repeats that are recognized by the Cre recombinase. Any DNA region flanked by two loxP sites (floxed) with the same orientation are excised following action of the Cre recombinase. Inactivation of ERα by deleting the DNA binding sequences and the weak dimerization domain that are contained in exon 3 abolishes the transcriptional activity of the receptor. Moreover, exon 3 contains several potential acceptor sequences that could result in the generation of an alternative splicing variant in case a region upstream of exon 3 was chosen for elimination. Finally, Cre-mediated deletion of exon 3 disrupts the reading frame of downstream ERα DNA sequences and inactivates the anti-apoptotic or any remaining activity of the receptor that requires the dimerization, and/or ligand binding domains.

### Generation of ERα Mutant Mice

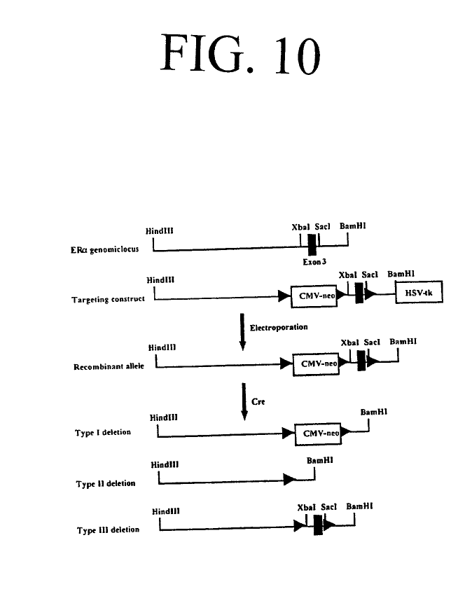

A 86 Kb genotropic locus containing exons 3 and 4 of the mouse ERα gene (FIG. 10), isolated from C57B1/6 mouse genotropic library and subcloned into a BAC vector, has been kindly provided by Dr. Ken Korach (NIH). This genotropic locus has been further characterized in terms of restriction mapping and sequence analysis and a HindIII/BamHI fragment containing exon 3 (0.2 Kb) flanked by a 5 kb left and a 3 kb right arm has been subcloned. The targeting construct is constructed in the pEasy-Flox vector, which contains 3 loxP sites, 2 of which are flanking a neomycin resistance (neo¹) gene under the control of the cytomegalovirus (CMV) promoter, and a HSV-thymidine kinase (HSV-tk) cassette. Depicted in FIG. 10 is the two step strategy for generating and targeting the floxed exon 3 in ES cells in vitro. In the first step the targeting construct which carries the floxed HSV-neo¹ selection marker upstream of the left arm, floxed exon 3 and the right arm and a subsequent HSV-tk cassette, is electroporated into C57B1/6 ES cells (obtained from Eurogentec, Belgium and tested positive by diploid aggregation and tetraploid aggregation for germline transmission) and introduced into the flanking regions of the target genotropic locus through homologous recombination.

Transfected ES cells are grown on a single layer of mitomycin C and LIF-treated, neo-resistant mouse embryonic fibroblasts from Swiss/129/Balb/c mice (also obtained from Eurogentec). Electroporation and selection of positive ES clones with G418 and ganciclovir is performed according to established protocols (Robertson et al. (1986) Nature 323: 445-448). Homologous recombination of the targeted construct into G418-positive clones is confirmed with Southern blot analysis of genotropic DNA. The frequency of homologous recombination has been reported to vary between 1 in 18 (Gu, H., Jet al. (1994) Science 265:103-106) and 1 in 50 (Pasparakis, M. et al. (1996) J Exp.Med. 184:1397-1411) double drug resistant colonies. In the second step, a plasmid carrying Cre under the control of the CMV promoter is elecroporated in the genetically modified ES cells. Because the expression of Cre is transient, the recombination event occurs only once between two of the three loxP sites, generating three possible types of deletion. Type I deletion results in the deletion of the complete exon 3-loxP-HSV-neo^{r} selection marker from the genome of ES cells and animals derived from the corresponding ES cells carry the floxed exon 3 in the germ line. Type III deletion is identified with Southern blot analysis. This approach has been described and successfully used for T -cell-specific deletion of the DNA polymerase β gene segment (Gu, H., Jet al. (1994) Science 265:103-106). Targeted ES cells are microinjectioned into BALB/C blastocysts. Male chimeras are mated to C57B1/6 females. Germ line transmission in the progeny is confirmed by Southern blot analysis. Finally, the functional integrity of the floxed gene is determined using classical receptor binding studies, as well as transcription assays using cells isolated from classical target tissues such as the ovaries and the uterus.

To obtain osteoblast or osteoclast-specific inactivation of ERα, Ind-αERKO mice are mated with the Tet-OG2-Cre transgenic mice or with TRAP-Cre transgenic mice. The derived offspring are grown to adulthood and, in the case of Tet-OG2-Cre, are then treated with Dox for a predetermined time period that allows for maximum Cre expression. Mice are then sacrificed, and tissues including calvariae, femurs, vertebrae, uteri, mammary glands, lungs, livers, kidneys, and brains are harvested, and genotropic DNA extracted. The efficiency of Cre-mediated cell specific deletion of exon 3 is evaluated by means of Southern blot analysis of the genotropic DNA extracted from the different tissues. Demonstration of osteoblast-specific inactivation of the ERα is accomplished by transfecting calvaria cells isolated from the ERα knockout mice with a multiple ERE-luciferase reporter construct. The specificity of ERα inactivation in osteoblastic, but not other cells, is established by performing the same study using, instead of calvaria osteoblasts, breast or uterine cells from the same mice. This approach has been successfully used previously by Ernst and colleagues to demonstrate the presence of low levels of functional endogenous ERs in osteoblastic cells (Ernst, M., M. G. Parker, and G. A. Rodan. (1991) Mol. Endocrinol. 11:1597-1606). In these experiments, potential effects on ERE transcription through the ERβ are eliminated by utilizing, instead of 17β-E₂, the ERα-specific ligand Propyl Pyrazole Triol, a synthetic compound developed by Dr. John Katzenellenbogen, University of Illinois, Urbana-Champaign. Propyl Pyrazole Triol is a structurally diverse ER ligand that is related to a group of compounds that have been reported by the Katzenellenbogen group (Sun, J., et al. (1999) Endocrinology 140:800-804). This compound has pronounced ERα-selective binding affinity (RBAs to 17β-E₂ are 63 for ERα versus 0.095 for ERβ) and high ERα transcriptional potency in transiently transfected human endometrial cancer (HEC-1) cells but no ERβ-mediated transcriptional activity even at the highest concentrations of treatment (personal communication). As an additional approach for demonstrating ERα inactivation in an osteoblast-specific manner in the knockout mouse, the knockout mouse is crossed with a ERE-lacZ reporter mouse. The derived litter and wild type animals are ovariectomized to remove endogenous estrogens. A week following ovariectomy, either type of mice is treated with Propyl Pyrazole Triol for 24 hours. Following the 24-hour treatment period, mice are sacrificed and transcriptional activity of ERα, or lack thereof, is assayed with β-galactosidase staining of uterine, breast and bone sections.

In addition to generating a mouse in which the ERα inactivation is targeted only to osteoblasts and osteoclasts, a different mouse in which ERα is inactivated in all cell types that express this gene is accomplished by crossing the Ind-αERKO mice with an existing transgenic mouse that carries the Mx-Cre transgene (Kuhn, Ret al. (1995) Science 269:1427-1429). In this mouse, the Cre recombinase is under the control of the Mx 1 gene promoter. This gene is silent in healthy mice, but it is turned on upon viral infection. Experimentally, the activity of the Mx1 promoter can be stimulated to very high levels by administration of 1FN-α or -β or a synthetic double-stranded RNA polyinosinic-polycytidylic acid (pI-pC), which is itself an IFN inducer, in mice. Mice harboring both the floxed ERα and the Mx-Cre acquire an inactive mutation in ERα upon treatment with IFN or pI-pC.

### EXAMPLE 10

### Method for Screening for Nongenotropic Estrogen like Signalling

Activators of nongenotropic estrogen like signalling which lack transcriptional activity are identified with a high throughput combination screening which simultaneously detects ERK activation using the SRE-SEAP detection system (positive readout) and C3 (complement 3) transcription (negative readout). To this end, HeLa cells are stably transfected using retroviruses with the hERα and/or hAR, an SRE-SEAP as well as a C3-Luc plasmid. In another aspect of the invention, stable transfectants in which the hERα or hAR gene are conditionally expressed under the control of the TET-OFF (tetracycline-OFF) system.

The SRE-SEAP assay detects activation of the MAPK/JNK signalling pathways by means of assaying transcription factor (Elk-1/SRF)—induced activation of a Serum Response Element (SRE) placed upstream of the herpes simplex virus thymidine kinase (HSV-tk) promoter that drives the expression of secreted alkaline phosphatase (SEAP). This assay is commercially available. Kits for in vivo assays for MEK1 or MEKK1 kinases are also commercially available. In general, 24 hrs after transfection serum-containing medium is replaced with medium that contains about 0.2% serum. Cells are culture for 24 hrs in about 0.2% serum and then assayed. Control samples are treated with vehicle alone.

Generally, HeLa cells are plated in 48-well plates at a concentration of 4-5×10⁴ per well and cultured overnight in phenol red-free MEM medium containing 10% charcoal-stripped FBS (fetal bovine serum), antibiotics (penicillin and streptomycin) and glutamine. The medium is removed, cells are washed twice in PBS and overlaid with serum-free and antibiotic-free medium that contains a transfection mix (lipofectamine, Lipofectamine plus and up to 0.6 mg of total amount of DNA to be transfected). Cells are incubated with the transfection mix for 2-3 hours. The transfection mix is removed and replaced with fresh phenol red-free MEM medium containing 10% charcoal-stripped FBS antibiotics and glutamine, and the cells are left to recover for 24 hours. The 10% FBS -containing medium is replaced with fresh phenol red-free MEM medium containing 0.2% charcoal-stripped FBS antibiotics and glutamine. Cells are cultured in 0.2% FBS-containing medium for 24 hours. Cells are treated with steroids at the desired concentration for 24 hours. SRE-SEAP activity is measured in 50 ml of the 0.2% FBS-containing cell culture medium with the commercially supplied kit and according to the manufacturer's instructions. In the case of transient transfections, data are corrected for transfection efficiency by assaying Renilla-luciferase activity in cell lysates.

17β-Estradiol is a positive compound control of ERK activation as well as C3 transcription. Pyrazole can be used as negative control of ERK activation and estren as a negative control of C3 transcription. Compounds with strong SRE-SEAP activity but weak or absent C3-Luc activity, compared to 17β-Estradiol are selected. The selection criteria can include classifying compounds of interest based on the ratio of ERK activation over C3 transcription. Compounds of interest are screened further for osteoblast/osteocyte anti-apoptotic activity.

Additionally, novel ligands using combinatorial chemistry and 3-D modeling of the binding pocket of the ER can be synthesized.

One skilled in the art will readily appreciate that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of inducing a selective effect of a receptor comprising contacting the receptor with a compound that induces a nongenotropic effect without substantially inducing a genotropic effect, wherein the receptor is selected from the group consisting of a progesterone receptor, a glucocorticoid receptor, a mineralcorticoid receptor, a retinoic acid receptor, vitamin D receptor, PPAR receptor, pregnane X receptor, bile acid receptor, thyroid receptor, farnesoid X receptor, liver X receptor, ecdysone receptor, and COUP-TF receptor.

2. The method of claim 1, wherein the nongenotropic effect occurs due to the interaction of the compound with the ligand binding domain of the receptor.

3. The method of claim 1, wherein the nongenotropic effect occurs due to the activation by the compound of a region of the receptor which is the functional equivalent of the ligand binding domain of the estrogen receptor.

4. The method of claim 1, wherein the genotropic effect is mediated by the DNA-binding domain of the receptor.

5. The method of inducing a selective effect of a receptor comprising contacting the receptor with the compound that induces a genotropic effect without substantially inducing a nongenotropic effect.

6. The method of claim 5, wherein the receptor is selected from the group consisting of a progesterone receptor, a glucocorticoid receptor, a mineralcorticoid receptor, a retinoic acid receptor, vitamin D receptor, PPAR receptor, pregnane X receptor, bile acid receptor, thyroid receptor, farnesoid X receptor, liver X receptor, ecdysone receptor, and COUP-TF receptor.

7. The method of claim 5, wherein the genotropic effect is protein synthesis.

8. A method for screening a compound that will induce a selective nongenotropic effect of a steroid receptor comprising
assessing the ability of the compound to activate a pathway mediated by the ligand-binding domain of the steroid receptor without substantially activating the DNA-binding domain of the steroid receptor or to activate the DNA-binding domain of the steroid receptor without substantially activating functions mediated by the ligand-binding domain of the steroid receptor; and then
measuring the biological activity of the test compound to assess its ability to induce a target nongenotropic effect without substantially inducing a genotropic effect or its ability to induce a target genotropic effect without inducing a substantial nongenotropic effect.

9. The method of claim 8, wherein the steroid receptor is not the estrogen or progesterone receptor.

10. The method of claim 8, wherein the steroid receptor is selected from the group comprising a progesterone receptor, a glucocorticoid receptor, a mineralcorticoid receptor, a retinoic acid receptor, vitamin D receptor, PPAR receptor, pregnane X receptor, bile acid receptor, thyroid receptor, famesoid X receptor, liver X receptor, ecdysone receptor, and COUP-TF receptor.

11. The method of inducing a selective effect of an estrogen receptor comprising contacting the estrogen receptor with a compound that induces a nongenotropic effect other than those mediated by the activation of extracellular regulated kinase without substantially inducing a genotropic effect.

12. The method of inducing a selective effect of an estrogen receptor comprising contacting the estrogen receptor with a compound that induces a nongenotropic effect for other than the treatment of bone loss or to increase bone mass without substantially inducing a genotropic effect.

13. The method of inducing a selective effect of an androgen receptor comprising contacting the androgen receptor with said compound that induces a nongenotropic effect other than those mediated by the activation of extracellular regulated kinase without substantially inducing a genotropic effect.

14. The method of inducing a selective effect of an androgen receptor comprising contacting the androgen receptor with a compound that induces a nongenotropic effect for other than the treatment of bone loss or to increase bone mass or for the treatment of an estrogen-related cancer or endometreosis without substantially inducing a genotropic effect.

15. The method of inducing a nongentropic effect of a steroid receptor, comprising administering a compound other than an estrogen or androgen containing a cyclopentanoperhydrophenanthrene nucleus which binds to the steroid receptor and induces a desired nongentropic effect while not inducing a steroid-induced genotropic effect.

16. A method for screening a compound to select a compound that induces a selective steroidal response comprising:
contacting a steroid receptor with a test compound;
determining whether a test compound activates the nongentropic activity of a receptor;
determining the level of transcriptional activity of the receptor-test compound complex; and
selecting the compound that activates the nongentropic activity of the receptor and induces transcriptional activity of the receptor less than 10% of endogenous steroid receptor ligands.

17. The method of claim 16, wherein the nongentropic activity is preventing apoptosis.

18. The method of claim 16, wherein the nongentropic activity is a signal transduction pathway.

19. The method of claim 16, wherein the nongentropic activity is a second messenger system.

20. The method of claim 18, wherein the signal transduction pathway is a protein kinase signal transduction pathway.

21. The method of claim 20, wherein the protein kinase signal transduction pathway is a MAP kinase signal transduction pathway.

22. The method of claim 21, wherein the MAP kinase signal transduction pathway is the Src/Shc/extracellular regulated kinase signal transduction pathways.

23. A method of evaluating a compound comprising the steps of:
growing a tissue culture cell line until cell growth has reached appropriate confluence;
transfecting the tissue culture with an expression plasmid of a specific receptor and an expression plasmid for a target gene responsive to the selected receptor;
contacting the transfected cells with a test compound;
determining whether the compound induces a nongenotropic effect without substantially inducing a genotropic effect.

24. The method of claim 23, wherein the receptor is not an estrogen or progesterone receptor.

25. The method of claim 23, wherein the target gene is selected from the group consisting of chloramphenicol acetyl transferase (CAT), β-galactosidase, alkaline phosphatase, luciferase, peptide hormones, growth factors and chimeric proteins.

26. The method of claim 23, wherein the level of transcription of the target gene is determined using a method selected from the group consisting of calorimetric, fluorescent; immunochemical, chemical or radiochemical methods.

27. A method for screening compounds for the treatment of steroid receptor related diseases or disorders comprising:
contacting a cell expressing a selected natural or artificial steroid receptor with a test compound;
assessing whether the compound activates a nongentropic activity of the steroid receptor;
determining the level of transcription induced by the test compound; and
selecting the compound or compounds that activate the nongentropic activity of the steroid receptor without substantially activating the genotropic activity of the steroid receptor.

28. A method for screening compounds for the treatment of steroid receptor related diseases or disorders comprising:
contacting a cell expressing a natural or artificial sex steroid receptor with a test compound alone or in combination with a pro-apoptotic agent;
assessing whether the compound activates a nongentropic activity of the steroid receptor;
determining the level of transcription induced by the test compound;
determining whether the test compound inhibited apoptosis in cells contacted with a test compound alone or in combination with a pro-apoptotic agent;
selecting the compound or compounds that activate the nongentropic activity of the steroid receptor without substantially activating the genotropic activity of the steroid receptor.

29. A method for screening for compounds that modulate the nongentropic activity of a steroid receptor without modulating the genotropic activity of the steroid receptor comprising the steps of:
contacting cells expressing a natural or artificial steroid receptor(s) with a test compound;
determining the amount of extracellular regulated kinase activation in the cells contacted with the test compound;
determining the amount of transcription in the cells contacted with the test compound;
selecting the cells contacted with the test compound that exhibit extracellular regulated kinase activation and minimal transcription levels as compared to transcriptions levels in cells contacted with an androgen or estrogen.

30. A method for screening for compounds that modulate the nongentropic activity of a steroid receptor without modulating the genotropic activity of the steroid receptor comprising the steps of:
contacting cells with at least one test compound wherein the cells contain:
(a) non-endogenous DNA which expresses a steroid receptor protein, or functional engineered or modified forms thereof,
(b) DNA which encodes an operative hormone response element linked to a first reporter gene; and
(c) DNA which encodes a second reporter gene operably linked to a signal transduction pathway responsive transcriptional control unit such that transcription of the second reporter gene is activated in response to activating a signal transduction pathway and wherein transcription of the second reporter gene is not activated by the steroid hormone;
assaying for evidence of transcription of the reporter genes in the cells; and
selecting the compounds that induce transcription of the second reporter gene and do not induce transcription of the first reporter gene.

31. A method for screening for compounds that modulate the nongentropic activity of a steroid receptor without modulating the genotropic activity of the steroid receptor comprising the steps of:
contacting cells with at least one test compound wherein the cells contain:
(a) non-endogenous DNA which expresses a steroid receptor protein, or functional engineered or modified forms thereof,
(b) DNA which encodes a reporter gene operably linked to a signal transduction pathway responsive transcriptional control unit such that transcription of the reporter gene is activated in response to activating a signal transduction pathway and wherein transcription of the second reporter gene is not activated by the steroid hormone;
assaying for evidence of transcription of the reporter gene in the cells;
quantifying the transcription of steroid receptor responsive genes; selecting the compounds that induce transcription of the reporter gene and do not induce transcription of steroid receptor responsive genes.

32. The method of claims 30 or 31, wherein the signal transduction pathway responsive transcriptional control unit is selected from the group consisting of serum response element, Activator protein 1, cAMP response element, E-box DNA binding element, E2F DNA binding element, glucocorticoid response element, heat shock response element, interferon γ activation sequence, interferon stimulated response element, nuclear factor of activated T cells, nuclear factor of κB cells, p53 response element, Rb response element, and STAT3 response element.

33. A bioassay for identifying a test compound or chemical signal that activates nongentropic receptor activity without substantially activating genotropic receptor activity, comprising the steps of:
growing a tissue culture screening system in a chemically defined culture medium, optionally supplemented with nutrients, wherein the cell line of the tissue culture screening system contains a steroid receptor having at least one identified nongentropic activity;
adding a test compound or chemical signal to the medium;
measuring the amount of transcription;
measuring the amount of nongentropic activity, selecting the compound that induces nongentropic activity but does not induce transcription.

34. The method of claim 33, wherein the cell line is OB-6.

35. A method of screening for ligands of steroid receptors that induce nongentropic activity without substantially inducing genotropic activity is provided comprising the steps of:
contacting a cell with a test compound wherein the cell has been transfected with:
i) a DNA sequence encoding a functional steroid receptor or genetic variant of a steroid receptor;
ii) a response element-reporter gene construct; and iii) serum response element-reporter gene construct;
determining the effect of the test compound on the transcription of the response element reporter gene construct;
determining the effect of the test compound on the transcription of the serum response element-reporter gene construct; and
selecting the compound that activates the transcription of the serum response element-reporter gene construct without substantially effecting the transcription of the response element reporter gene construct.

36. A method of screening for ligands of steroid receptors that induce nongentropic activity without substantially inducing genotropic activity is provided comprising the steps of:
contacting a first cell with a test compound wherein the first cell has been transfected with:
i) a DNA sequence encoding a functional steroid receptor or genetic variant of a steroid receptor;
ii) a response element-reporter gene construct; determining the effect of the test compound on the transcription of the response element reporter gene construct;
contacting a second cell with the test compound wherein the second cell has been transfected with a signal transduction pathway responsive transcriptional control unit operably linked to a reporter gene;
determining the effect of the test compound on the transcription of the second cell; and
selecting the compound that activates the transcription of the second cell without substantially effecting the transcription of the first cell.

37. A kit for measuring steroid receptor dependent nongentropic activity comprising: a container having a stable transfected cell line wherein the cell line includes a steroid responsive reporter gene construct, a means for assessing the activation of nongentropic steroid activity, and a steroid receptor.
